(19) United States
(12) Patent Application Publication
(10) Pub. No.: US 2026/0192155 A1
(43) Pub. Date: Jul. 9, 2026

(54) SYSTEMS AND METHODS OF PROVISIONING A FEEDBACK BASED ON AN ACTIVITY

(71) Applicant: Neotericc LLC, LONE TREE, CO (US)

(72) Inventor: Patrick L. Carter, Lone Tree, CO (US)

(73) Assignee: Neotericc LLC, LONE TREE, CO (US)

(21) Appl. No.: 19/435,961

(22) Filed: Dec. 30, 2025

Related U.S. Application Data
(60) Provisional application No. 63/739,828, filed on Dec. 30, 2024.
(60) Provisional application No. 63/739,835, filed on Dec. 30, 2024.
(60) Provisional application No. 63/742,862, filed on Jan. 8, 2025.

(51) Int. Cl. A63B 24/00; A63B 71/06

(52) U.S. Cl. CPC A63B 24/0062; A63B 24/0021; A63B 71/0622; A63B 71/0669; A63B 2024/0034; A63B 2220/05; A63B 2220/806; A63B 2220/836; A63B 2225/20; A63B 2225/50

(57) ABSTRACT

The present disclosure provides a method of provisioning a feedback based on an activity. Further, the method may include receiving, using a communication device, an activity data associated with a performance of the activity from a data source device. Further, the receiving may be performed based on a real-time communication protocol. Further, the method may include generating, using a processing device, a feedback data using an artificial intelligence (AI) module based on the activity data. Further, the method may include transmitting, using the communication device, the feedback data to a user device associated with a user. Further, the transmitting may be performed based on the real-time communication protocol.

### REFERENCE TO RELATED APPLICATIONS

This application claims the benefit of U.S. Provisional Ser. No. 63/739,828, titled “METHODS AND SYSTEMS OF PROVISIONING A FEEDBACK BASED ON AN ACTIVITY”, filed on Dec. 30, 2024; U.S. Provisional Ser. No. 63/739,835, titled “SYSTEMS AND METHODS OF PROVISIONING A 3D MODEL DATA BASED ON AN ACTIVITY”, filed on Dec. 30, 2024; and U.S. Provisional Ser. No. 63/742,862, titled “SYSTEMS AND METHODS OF PROVISIONING AN INTEGRATED BIOMECHANICAL DATASET”, filed on Jan. 8, 2025, each of which is incorporated by reference herein in its entirety.

### FIELD OF DISCLOSURE

The present disclosure relates to the field of data processing. More specifically, the present disclosure relates to systems and methods of provisioning a feedback based on an activity.

### BACKGROUND

The field of sports performance analysis, biomechanical evaluation, and technology-enabled coaching, and more particularly to systems and methods that support the assessment, interpretation, and communication of athletic performance data is increasingly important due to the growing reliance on data-driven decision-making in sports training, athlete development, injury prevention, rehabilitation, and remote coaching environments. As athletic performance becomes more competitive and globally distributed, there is a rising demand for technological solutions that may support precise evaluation, timely feedback, and scalable coaching across diverse settings, including professional sports, amateur training, youth development, and clinical rehabilitation.

A desirable objective in the given field is to enable accurate, timely, and context-aware evaluation of athletic performance that may support effective instruction, coaching, and decision-making across both live and recorded scenarios. Such an objective includes the ability to assess performance, to interpret complex motion patterns and interactions, to present insights in forms that are meaningful to different stakeholders, and to support collaboration between geographically distributed participants. Achieving the said objective is critical for improving training outcomes, optimizing skill acquisition, reducing the risk of injury, and enabling equitable access to high-quality coaching resources.

However, existing approaches in the given field face a number of technical and practical limitations that hinder the achievement of the said objective. Many existing systems rely on fragmented data sources that are analyzed in isolation, increasing the difficulty to derive a holistic understanding of performance. Systems that process motion or video data often struggle with temporal accuracy, resulting in delayed or misaligned feedback that reduces instructional effectiveness. In addition, existing solutions frequently lack the ability to adapt analysis and feedback to the specific context, role, or historical performance of an individual user, leading to generalized outputs that may not address individual needs.

Further challenges arise in environments that require real-time interaction or remote collaboration. Existing tools often provide limited support for synchronized interaction between multiple participants, particularly when performance data, visualizations, and feedback must be shared consistently across distributed devices and network conditions. Asynchronous review mechanisms, when available, are frequently disconnected from live sessions, creating discontinuities between instruction, practice, and follow-up analysis. Moreover, the increasing volume and complexity of performance-related data may overwhelm users, increasing the difficulty to extract actionable insights without significant manual interpretation.

Additional problems are encountered when attempting to support diverse modes of interaction and communication. Many systems are constrained to a single mode of output, such as numerical reports or static visualizations, which may not align with user preferences or situational requirements. The lack of integrated support for conversational interaction, dynamic visualization, and adaptive feedback further limits usability and effectiveness. The said limitations are compounded in scenarios that involve long-term performance tracking, where gradual changes or emerging issues may go unnoticed due to insufficient longitudinal analysis capabilities.

Therefore, there is a need for improved systems and methods of provisioning a feedback based on an activity that may overcome one or more of the preceding problems.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a method of provisioning a feedback based on an activity. Further, the method may include receiving, using a communication device, an activity data associated with a performance of the activity from a data source device. Further, the receiving may be performed based on a real-time communication protocol. Further, the method may include generating, using a processing device, a feedback data using an artificial intelligence (AI) module based on the activity data. Further, the method may include transmitting, using the communication device, the feedback data to a user device associated with a user. Further, the transmitting may be performed based on the real-time communication protocol.

The present disclosure provides a system of provisioning a feedback based on an activity. Further, the system may include a communication device. Further, the communication device may be configured for receiving an activity data associated with a performance of the activity from a data source device. Further, the receiving may be performed based on a real-time communication protocol. Further, the communication device may be configured for transmitting a feedback data to a user device associated with a user. Further, the transmitting may be performed based on the real-time communication protocol. Further, the system may include a processing device communicatively coupled with the communication device. Further, the processing device may be configured for generating the feedback data using an artificial intelligence (AI) module based on the activity data.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

The drawings presented with this disclosure may illustrate representative and non-limiting arrangements of hardware components, software modules, artificial intelligence subsystems, machine learning architectures, data processing pipelines, user interfaces, network topologies, and memory arrangements that may be used to understand embodiments of the present subject matter. They may depict functional or conceptual layouts intended to facilitate explanation of the disclosed principles. The geometric appearance, dimensional proportions, ordering, grouping, and naming of elements within the drawings are not intended to imply any restriction on implementation. The drawings may schematically portray computing environments containing client devices, servers, distributed computing clusters, communication networks, storage systems, or artificial intelligence models arranged for training, inference, or combined operations. The drawings may include simplified symbolic representations of algorithmic processes, workflows, blocks, or modules; such symbolic representations are treated as abstractions of underlying hardware and software operations rather than literal structural requirements. Similarly, lines connecting components may represent logical associations, communication pathways, or data relationships rather than any specific physical wiring or layout. These figures may also illustrate non-exhaustive examples of operational stages, sequencing, or interactions among artificial intelligence components such as encoders, decoders, generators, discriminators, featurizers, transformers, or safety-validation modules. Any specific combination or configuration shown is presented for explanatory clarity only. Additional drawings, alternative views, or more granular depictions may be used without affecting the scope of the claims.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 is a block diagram illustrating a machine-learning system 300 for implementing various embodiments of this disclosure, in accordance with some embodiments.

FIG. 4 illustrates a flowchart of a method 400 of provisioning a feedback based on an activity, in accordance with some embodiments.

FIG. 5 illustrates a block diagram of a system 500 for provisioning a feedback based on an activity, in accordance with some embodiments.

FIG. 6 is an illustration of a user interface 600 of a user presentation device showing an integration of one or more of scorecards, video overlays, and real-time communication technologies for feedback delivery, in accordance with some embodiments.

FIG. 7 illustrates a three-dimensional top down view during a release phase in a sport of basketball, in accordance with some embodiments.

FIG. 8 illustrates a user presentation device 800 showing a remote, collaborative coaching platform, in accordance with some embodiments.

FIG. 9 illustrates a flowchart of a method 900 of provisioning an integrated biomechanical dataset, in accordance with some embodiments.

FIG. 10 illustrates a flowchart of a method 1000 of provisioning an integrated biomechanical dataset including receiving, using the communication device 1102, the additional information data from the user device 1110 associated with the user, in accordance with some embodiments.

FIG. 11 illustrates a block diagram of a system 1100 of provisioning an integrated biomechanical dataset, in accordance with some embodiments.

FIG. 12 illustrates an operational workflow 1200 of the system 1100 for provisioning an integrated biomechanical dataset, in accordance with some embodiments.

FIG. 13 illustrates a flowchart of a method 1300 for provisioning a three-dimensional model data based on an activity, in accordance with some embodiments.

FIG. 14 illustrates a flowchart of a method 1400 for provisioning a three-dimensional model data based on an activity including retrieving, using the storage device, the 3D model data, in accordance with some embodiments.

FIG. 15 illustrates a flowchart of a method 1500 for provisioning a three-dimensional model data based on an activity including receiving, using the communication device 1602, the modified 3D data from the user device 1608 associated with the user, in accordance with some embodiments.

FIG. 16 illustrates a block diagram of a system 1600 for provisioning a three-dimensional model data based on an activity, in accordance with some embodiments.

FIG. 17 illustrates a user presentation device 1700 presenting a real-time 3D skeleton rendering and adjustment tools during a collaborative coaching session, in accordance with some embodiments.

FIG. 18 illustrates a flowchart of a method 1800 for provisioning a three-dimensional model, in accordance with some embodiments.

FIG. 19 illustrates a block diagram of a system 1900 for provisioning a three-dimensional model, in accordance with some embodiments.

FIG. 20 illustrates a flowchart of a method 2000 for facilitating dataset creation and indexing, in accordance with some embodiments.

FIG. 21 illustrates a flowchart of a method 2100 for facilitating dataset creation and indexing including retrieving, using the storage device 2306, at least one relevant dataset portion from the integrated biomechanical dataset using at least one of a vector similarity and a graphical constraint, in accordance with some embodiments.

FIG. 22 illustrates a flowchart of a method 2200 for facilitating dataset creation and indexing including generating, using the processing device 2304, at least one anonymized dataset portion, in accordance with some embodiments.

FIG. 23 illustrates a block diagram of a system 2300 for facilitating dataset creation and indexing, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

The detailed description that follows may provide a framework for describing computer-implemented systems, artificial intelligence systems, distributed learning infrastructures, data processing pipelines, and hardware and software arrangements suitable for implementing embodiments shown in the drawings. Terms such as processing, computing, determining, or generating refer to actions performed by computing systems or electronic devices that manipulate data represented as physical signals, stored values, or encoded information within registers, memory structures, and storage devices.

The present disclosure contemplates implementations involving artificial intelligence, machine learning, distributed computation, and computer-implemented systems operating upon data represented as physical electronic or optical signals. Descriptions of processing, analyzing, determining, transforming, encoding, decoding, generating, inferring, synthesizing, modifying, storing, retrieving, ranking, filtering, validating, classifying, or otherwise manipulating information are to be understood as referring to the actions of computing systems, electronic devices, or computational circuits that manipulate such signals in memory elements, registers, buffers, or storage media. These operations may be performed by general-purpose processors, specialized processors, machine learning accelerators, or combinations thereof.

The disclosure contemplates implementations in which artificial intelligence systems perform perception, synthesis, inference, prediction, or generation of information using models whose configurations may evolve based on training, feedback, or adaptive learning processes. A model may initially be configured with a set of parameters and architectural structures that define its behavior, and this configuration may change automatically as the model encounters training inputs, validation data, reference data, or instructor-provided feedback. A machine learning model may modify its internal state through optimization techniques, gradient updates, reinforcement signals, vector transformations, attention mechanisms, latent variable adjustments, embedding refinements, or other learning operations executed electronically. Such modifications may occur over extended cycles, partial cycles, or continual learning sequences without explicit intervention by a human.

The disclosure contemplates systems involving data ingestion pipelines that gather input from sources including but not limited to sensor signals, event streams, text data, image data, audio data, video data, structured and unstructured repositories, application logs, telemetric feeds, network services, or human-generated content. Ingestion functions may include filtering, normalization, augmentation, segmentation, batching, tokenization, windowing, compression, encryption, decryption, hashing, deduplication, contextualization, and mapping to internal formats. Intermediate components may transform this data into derived representations, including embeddings, latent encodings, feature tensors, multi-modal joint representations, or contextual vectors suitable for use by downstream modeling engines. These transformations may be performed using neural networks, statistical encoders, dimensionality-reduction algorithms, or hybrid computational modules.

The disclosure contemplates machine learning systems that may employ advanced architectures such as transformer networks, encoder-decoder stacks, mixture-of-experts structures, diffusion models, recurrent networks, convolutional hierarchies, attention-based models, retrieval-augmented architectures, cross-modal alignment engines, graph neural networks, probabilistic models, auto-encoding frameworks, or hybrid symbolic-neural systems. Such models may implement deep layers configured to perform operations including attention calculations, feed-forward projections, gating operations, positional encoding, normalization steps, multi-head routing, sequential decoding, or latent pathway selection. Multi-modal systems may combine textual, visual, auditory, sensory, or structured inputs within joint representational spaces. Embeddings may be learned from large corpora or multi-modal datasets and may encode semantic, syntactic, structural, temporal, spatial, or contextual relationships across modalities. These embeddings may be dynamically updated as the system encounters new information, thereby improving consistency, expressiveness, or alignment with real-world contexts.

The disclosure contemplates training processes that may involve supervised learning, unsupervised learning, semi-supervised learning, self-supervised learning, reinforcement learning, preference optimization, curriculum-based learning, active learning, or continual learning. Training operations may include forward passes through the model, backward propagation of gradients, update steps using optimization algorithms, adaptive learning-rate scheduling, regularization steps, loss-function evaluation, and check pointing of intermediate states. Training datasets may include real-world data, synthetic data, simulated data, augmented data, or mixtures thereof. Validation procedures may evaluate performance metrics, generalization behavior, safety constraints, or compliance with domain-specific criteria. In some implementations, refinement cycles may incorporate human-in-the-loop interventions, reward model shaping, safety evaluator feedback, or guided corrections.

The disclosure contemplates distributed or federated execution in which computation is partitioned across multiple hardware devices, regions, or clusters. Certain operations may occur at edge devices for low latency, while others may be delegated to remote servers, cloud clusters, datacenters, or specialized compute fabrics. Components may communicate over wired or wireless networks supporting data exchange, synchronization, replication, or model-state updates. Distributed learning processes may synchronize gradients, coordinate model versions, merge updates across shards, or exchange activation values within parallel training regimes. Distributed inference may involve routing requests across replicas, balancing load through orchestration layers, or selecting model pathways dynamically. Network connections may include encryption, authentication, secure session management, or routing protocols appropriate for maintaining privacy, integrity, or availability.

The disclosure contemplates orchestration layers capable of managing complex workflows involving model invocation, tool invocation, external data retrieval, decision routing, fallback selection, multi-model aggregation, post-processing evaluation, or safety governance. Orchestration environments may evaluate contextual signals, metadata, user characteristics, or policy constraints to determine which models, subsystems, or computational branches should be executed. Such environments may dynamically alter execution pathways based on estimated performance, resource availability, model confidence, safety risk, or real-time system health. Post-processing components may evaluate generated outputs for compliance with content policies, statutory requirements, operational constraints, or domain-specific decision rules.

The disclosure contemplates safety-oriented components that evaluate model outputs or intermediate representations for consistency with safety criteria, quality thresholds, regulatory considerations, factual accuracy constraints, domain restrictions, or alignment requirements. Safety modules may employ auxiliary models, discriminators, rule sets, statistical detectors, confidence estimators, or hybrid evaluators to identify undesirable outputs. These modules may trigger remediation actions including output modification, output rejection, re-routing through alternate inference pathways, invocation of corrective models, or escalation for human review. Safety processes may incorporate real-time validation, contextual scoring, adversarial robustness analysis, anomaly detection, or controlled generation constraints.

The disclosure contemplates governance structures including policy managers, audit loggers, compliance trackers, version controllers, and provenance systems that associate model outputs with contextual metadata, historical signals, update events, training sources, or safety evaluations. Systems may maintain lineage records documenting which model version, configuration state, or training dataset contributed to an outcome. Governance modules may ensure that system behavior aligns with formal requirements such as fairness principles, legal obligations, industry standards, or institutional guidelines.

The disclosure contemplates storage and memory systems capable of storing model parameters, datasets, embeddings, logs, metrics, checkpoints, execution traces, and auxiliary information used to configure or interpret model behavior. These storage systems may include magnetic media, semiconductor memory, optical media, solid-state arrays, distributed storage fabrics, or hybrid memory hierarchies. Storage media may contain instructions, configurations, or data structures that, when accessed by a computing device, configure that device to carry out the operations described herein. Such media may include executables, bytecode, machine code, firmware, microcode, program modules, configuration files, architectural descriptors, or schema definitions.

The disclosure contemplates user interfaces that permit human operators to view model outputs, initiate tasks, modify configurations, inspect metrics, interact with logs, evaluate safety signals, or guide system adaptation. Interfaces may be multimodal and may support textual input, speech commands, visual interaction, gesture control, or programmatic invocation through APIs. Administrative interfaces may allow for reviewing system performance, tuning operational thresholds, enabling or disabling features, monitoring resource use, examining generated content, or initiating refinement workflows.

The disclosure contemplates systems in which instructions are executed entirely on a single device, partially on multiple devices, or cooperatively across remote and local environments. Code may execute directly on hardware, within firmware, inside virtual machines, inside containers, or through any combination of software and hardware interactions. Computational instructions may be stored locally, transferred via communication networks, or streamed from remote systems. Implementations may involve software executing on general-purpose processors, specialized logic circuits performing equivalent functions, or hybrid mechanisms that combine hardware acceleration with software guidance.

Interpretation of terms in this disclosure is governed by principles commonly applied by persons of ordinary skill in the relevant field. Technical and scientific terms used herein should be understood in a manner consistent with their usage in the field of artificial intelligence, machine learning, computing, networking, data storage, or any related discipline. Terms describing functionality should not be interpreted as strictly structural unless explicitly stated. Phrases such as configured to, adapted to, operable to, or capable of indicate permissible functionality rather than structural limitations. Terms such as a or an encompass one or more unless clearly contradicted by context. Terms joined by or should be interpreted as inclusive, and terms joined by and should be interpreted as collective.

The description set forth herein provides a broad and flexible framework intended to support a wide range of computer-implemented, machine-learning-enabled, distributed, and multimodal embodiments. Variations may include reallocation of tasks, substitution of algorithms, reconfiguration of models, changes to pipeline ordering, or adoption of alternate hardware. No combination or arrangement mentioned herein should be regarded as required unless explicitly stated. The scope of protection is established by the claims, interpreted in light of this description.

The present disclosure contemplates implementations that employ advanced mathematical frameworks characteristic of modern artificial intelligence systems. Machine learning models may be conceptualized as parameterized functions that map elements of an input space to elements of an output space. Such a function may be defined over real-valued, complex-valued, vector-valued, tensor-valued, or mixed-modal domains. The model may implement successive transformations applied to an ordered set of input vectors using compositions of linear operators, nonlinear activations, attention functions, normalization operations, and dimensional projections.

Model parameters may be represented as ordered collections of real-valued scalars arranged into structures such as matrices, tensors, kernels, filters, or embeddings. These parameters may be optimized by minimizing a loss functional defined over an expected distribution of input-output pairs. The optimization process may involve computing gradients of the loss functional with respect to each model parameter, followed by an update step that serves to reduce the value of the loss functional. Gradient computation may use automatic differentiation frameworks that symbolically or numerically propagate partial derivatives backward through a computational graph.

Attention mechanisms may employ a similarity measure between projected query vectors and projected key vectors. This similarity measure may yield a weight distribution over contextual elements. The weighted combination of projected value vectors may form an attention output that is subsequently transformed through additional layers. Multiple independent attention heads may be aggregated to capture heterogeneous relationships within the input domain. Cross-attention mechanisms may operate similarly but with distinct source and target sequences.

Normalization steps may rescale intermediate representations using learned scaling and shifting coefficients. Activation functions may introduce nonlinearity by applying element-wise transformations selected to ensure differentiability and expressive capacity. Residual pathways may combine transformed and untransformed representations to facilitate stable gradient propagation under deep compositions. Positional encodings or structural embeddings may inject ordering, spatial, temporal, or relational information into otherwise permutation-invariant architectures.

Multi-modal models may operate over domains that combine text, image, audio, video, sensor, or structured signals. These domains may be embedded into a common vector space through learned projection operators. Joint training processes may enforce alignment constraints that minimize representational divergence between modalities while preserving intra-modal semantics.

Diffusion frameworks may model data generation as the reversal of a stochastic corruption process. A forward process may incrementally add noise to data samples, while a learned reverse process may approximate the time-reversed conditional probability distribution. The reverse process may be parameterized by a neural network trained to denoise intermediate states. Continuous-time formulations may model this process using stochastic differential equations whose drift and diffusion terms are learned through score-matching or related techniques.

Reinforcement-based procedures may model learning as an optimization of expected reward under a policy function. The policy may produce distributions over actions given a latent or explicit representation of the environment state. Policy gradients may be estimated from sampled trajectories, and advantage estimators may reduce the variance of such gradients. Value functions may approximate the expected cumulative reward, and these approximations may be updated through temporal-difference learning.

Generative models may be expressed in probabilistic terms as joint or conditional distributions parameterized by neural architectures. Such models may perform sampling by iteratively drawing latent variables from a learned distribution and transforming those variables into output space. Variational models may introduce auxiliary latent variables whose posterior distributions are approximated through recognition functions that optimize an evidence-bound objective.

Matrix decompositions, spectral analysis, manifold learning, kernel operators, and other mathematical constructs may be incorporated to improve expressiveness, stability, or computational efficiency. Training may involve sophisticated schedulers, trust-region constraints, adaptive learning-rate schemes, gradient-norm clipping, regularization penalties, entropy maximization, attention masking, or mixed-precision arithmetic.

All such mathematical constructs are conceptual, descriptive, and non-limiting. The disclosure encompasses any differentiable or non-differentiable optimization method, any discrete or continuous learning paradigm, and any representational transformation that may be understood by a person of ordinary skill in the field.

Further, the disclosure provides a computing environment which may include a combination of client devices, servers, distributed computing clusters, databases, external data sources, network nodes, and interface endpoints. Such an environment may support artificial intelligence workloads including perception, synthesis, inference, prediction, and generation, using hardware and software foundations designed for high-throughput and low-latency operation. Embodiments may involve the coordinated use of multiple machine learning models, whose configurations may evolve over time as they learn from training, validation, reference, or feedback data. Models may adjust their internal parameters through supervised, unsupervised, or reinforcement-based processes, allowing automatic electronic improvements to their performance based on input data and observed outcomes.

Further, the disclosure provides a computing device which may include processing units, memory elements, storage devices, system buses, high-speed controllers, low-speed controllers, and expansion interfaces. Processors may include general-purpose units, multi-core processors, vector processors, digital signal processors, tensor accelerators, neural accelerators, graphics engines, or various kinds of specialized integrated circuits including FPGAs, ASICs, ASSPs, SoCs, and CPLDs. A device may include system memory composed of volatile or non-volatile components such as RAM, DRAM, flash memory, ROM, or phase-change memory. The storage subsystem may include solid-state drives, magnetic disks, optical media, arrays of storage devices, and network-attached storage resources. Input and output mechanisms may include microphones, displays, keyboards, pointing devices, biometric sensors, gesture or touch interfaces, and actuators suitable for multimodal interaction with a user.

Further, the disclosure provides a machine-learning architecture which may include engines or modules such as a data input engine, data retrieval engine, data transform engine, featurization engine, modeling engine, generative engine, validation engine, feedback engine, and refinement engine. A data input pipeline may obtain structured or unstructured information from various sources, transform the information into model-compatible forms, and store such transformed data in memory or storage accessible to downstream components. A modeling engine may perform tasks such as model training, re-configuration, validation, and testing, executing iterative processes across multiple cycles or passes through training data. A predictive or generative engine may construct outputs based on intermediate representations, learned embeddings, or latent encodings generated by layers such as encoder-decoder structures, attention mechanisms, or multi-layer transformer architectures. Embeddings may represent discrete entities such as words, documents, or images as continuous vectors in high-dimensional spaces, capturing semantic or structural relationships useful for downstream tasks.

Further, the disclosure may provide a distributed or cloud-based operation may include multiple physical or virtual instances of computing devices, distributed across data centers or network boundaries. Functions may be partitioned across machines to achieve parallelism, redundancy, fault tolerance, or improved throughput. Distributed systems may use load balancing mechanisms to maintain stable processing, memory, or bandwidth utilization across clusters and avoid overload conditions. Such deployments may require communication over wired or wireless networks that implement a variety of protocols including HTTP, HTTPS, MQTT, CoAP, or any other suitable communication framework. Communication channels may include local networks, wide-area networks, personal-area networks, or global communication systems, potentially utilizing secure encrypted sessions such as SSL-based channels.

Further, the disclosure may provide an algorithm, process, or flow diagram which may include operations that may occur in sequences, reversed orders, concurrently, or in partially overlapping timelines, depending on the implementation. Blocks representing actions in a flowchart may correspond to program modules, instruction sequences, or hardware logic capable of performing the specified acts. Such operations may manipulate physical quantities such as electrical or magnetic signals stored or transferred among memory units, registers, storage devices, or communication media. Flow diagrams may be realized through software running on general-purpose processors, through dedicated hardware circuits, or through combinations of both.

Further, the disclosure may provide memory, storage, or programmatic constructs which may include program instructions encoded on computer-readable media including electronic, magnetic, optical, electromagnetic, semiconductor, or other tangible media. Examples include RAM, ROM, EEPROM, flash memory, magnetic disks, optical disks, and mechanical encoded structures such as punch cards or raised-pattern media. Such storage media may store instructions that, when executed, configure the memory and therefore configure the computing device itself, causing the device to perform functions described in association with the drawings.

Further, the disclosure may provide a user interface which may include graphical displays, dashboards, selection controls, input fields, monitoring elements, or multimodal interaction surfaces, allowing users to interact with computing systems in speech, touch, gesture, or other modalities. Such interfaces may be presented through client devices, server applications, or remote access platforms and may support visualization of model behavior, systems performance, or configuration parameters.

Further, described features may be combined, rearranged, omitted, or substituted without departing from the principles disclosed. Variations may involve distributing functionality across devices, merging components, implementing features in hardware rather than software, or employing alternative communication protocols. Many such variations and modifications are intended to fall within the scope of the disclosure as understood by persons skilled in the art.

The detailed description of the drawings therefore provides a foundation for describing technical, architectural, and operational aspects of embodiments, while allowing broad flexibility in how such embodiments may be implemented in practice. The scope of such embodiments is governed by the claims rather than the illustrative content of the drawings.

In some embodiments, a system consistent with this disclosure includes one or more client devices, one or more servers, and one or more data stores coupled by one or more networks. The client devices can include, without limitation, mobile phones, tablet computers, laptop or desktop computers, wearable devices, smart displays, vehicles, robots, or other computing platforms equipped with data processing hardware and memory hardware. The servers can include data servers, application servers, web servers, proxy servers, or cloud computing services that provide shared processing, storage, and networking resources. The data stores can include databases, object stores, file systems, or other repositories that persist configuration data, training data, logs, model artifacts, and other information.

The networks can include public and private networks, such as local area networks, wide area networks, and cloud networks, using wired or wireless communication links. The networks can provide routing, addressing, access control, encryption, and related functionality using standard or proprietary protocols.

Each computing device, whether a client device or a server, can include one or more processors, system memory, persistent storage, communication interfaces, and input or output devices. The processors can include general purpose central processing units, graphics processing units, digital signal processors, microcontrollers, application specific integrated circuits, field programmable gate arrays, or other programmable or fixed function processing elements configured to execute instructions or perform logic operations. The memory can include volatile and non-volatile storage, such as random access memory and read only memory. The persistent storage can include solid state drives, magnetic disks, optical media, or other non-transitory computer-readable media.

Program code executed by the processors can include operating systems, device drivers, libraries, and application programs, including components that implement portions of the methods described herein. Program code and data can be stored on computer-readable media and loaded into memory by standard mechanisms, such as boot loaders, installation programs, or update services.

Input devices can include keyboards, pointing devices, microphones, cameras, touch-sensitive surfaces, biometric sensors, and other sensors. Output devices can include displays, speakers, haptic devices, printers, and other actuators. Some devices can support multimodal interaction, allowing combined or sequential input and output through various modalities.

For purposes of this disclosure, artificial intelligence systems may include arrangements of software and hardware that perform tasks such as perception, prediction, planning, or generation based on input data. These systems can employ one or more models, such as statistical models, neural networks, decision trees, or other machine learning models. As used herein, a “model” can refer to a parameterized function, an ensemble of such functions, or a collection of cooperating components that process data and produce outputs.

In some embodiments, the system includes a data input engine that obtains data from one or more sources, such as application logs, sensor streams, structured databases, and unstructured content. The data input engine can retrieve, filter, aggregate, or transform the data into feature representations suitable for model consumption. Data sources can include training data, validation data, and reference data used to evaluate and calibrate model behavior.

A modeling engine can manage one or more training processes for one or more models. The modeling engine can select model architectures, initialize parameters, and apply training algorithms such as supervised learning, semi supervised learning, unsupervised learning, reinforcement learning, or combinations thereof. The modeling engine can also manage hyper parameters, training schedules, and evaluation procedures across epochs or passes through the data.

The system can include a generative response engine or inference engine that receives prompts or other inputs and generates outputs using one or more models. For example, a natural language interface can receive a text prompt, embed or otherwise encode the prompt, process the encoded prompt using a transformer based model or other sequence model, and generate a sequence of tokens that are decoded into an output. The engine can generate multiple candidate outputs and apply validation or ranking logic to select a final result according to quality, safety, or relevance criteria.

A feedback engine can collect explicit or implicit feedback signals, such as user ratings, corrective edits, or outcome metrics derived from downstream tasks. A refinement engine can use such feedback to adjust model parameters, routing logic, or policies, for example by performing additional training steps, updating reward models, or modifying configuration parameters.

In certain embodiments, the disclosed techniques are applied to platforms that include sensors and actuators, such as vehicles, robots, or other machines. The platform can include a processor system that receives signals from cameras, LIDAR units, radar units, inertial sensors, and other devices, and produces control outputs for steering, propulsion, braking, or other actuators. Sensor data can be captured at various sampling rates and processed by perception models to detect and track objects and infer scene attributes.

Planning and control components can receive outputs from perception models along with route information, traffic rules, and high-level goals. These components can generate trajectories or control commands, optionally using reinforcement learned policies, optimization-based planners, or hybrid systems. Connections to backend services can permit off-board processing, fleet-level learning, or remote supervision where appropriate, while on-board components can maintain safe operation in the presence of network latency or failures.

The systems described herein can be implemented using centralized, decentralized, or hybrid arrangements. For instance, models may be deployed in cloud environments, on edge devices, or across both, depending on requirements such as latency, privacy, cost, and reliability. Load balancing and resource management components can distribute processing across devices or data centers and can provide elasticity to accommodate changing workloads.

Certain embodiments may expose functionality through application programming interfaces, software development kits, or graphical user interfaces. Client applications can submit requests to backend services, which can apply authentication, authorization, logging, and policy enforcement before invoking models or tools and returning results.

The systems and methods disclosed herein can be implemented in hardware, software, firmware, or any combination thereof. In some embodiments, operations are carried out by one or more processors executing program instructions stored on one or more non-transitory computer-readable media. Such media can include, without limitation, semiconductor memory, magnetic storage, optical storage, and combinations thereof. Program instructions, when executed by the processors, cause the processors to perform the operations described herein.

Instructions can be delivered to computing devices in various ways, such as pre-installation, physical distribution of media, or transmission over networks. Instructions received over a network can be stored in memory or persistent storage and then executed by one or more processors. Dedicated hardware logic, such as application specific integrated circuits or field programmable gate arrays, can be used alone or in combination with software to implement certain functionality.

Any methods described in connection with embodiments of the present disclosure can be represented as one or more flow diagrams or state diagrams. Blocks in such diagrams can correspond to modules, components, operations, or code segments that implement the associated functionality. Blocks can be reordered, combined, executed concurrently, or omitted according to implementation specific considerations, unless a particular ordering is required by the claims.

Examples and embodiments described herein illustrate, rather than limit, the claimed subject matter. Certain features have been described in connection with particular embodiments for clarity, but other embodiments can include such features in different combinations. Features described in separate embodiments can be combined, and features described in a single embodiment can be separated, unless such combinations or separations are inconsistent with the claims. The scope of the disclosure is defined by the claims and their equivalents.

### Overview

The present disclosure provides methods and systems for real-time feedback using adaptive AI scoring models and personalized instruction based on individual performance. The platform supports multiple modes for delivering feedback, including scorecards, video overlays, and live collaborative coaching sessions through WebRTC or similar real-time communication frameworks. The system leverages temporal data analysis to generate in-the-moment feedback, seamlessly synchronizing insights with video streams for enhanced performance evaluation.

In some embodiments, the present disclosure provides the following features:
1. A system that delivers real-time feedback through scorecards, overlays, and AI-driven personalized instruction.
2. Integration of WebRTC or similar real-time communication technologies for collaborative coaching and peer-to-peer sports sessions.
3. A method for generating personalized insights using adaptive scoring models based on temporal data and performance phases.
4. Synchronization of AI-generated feedback with live or recorded video streams to enhance instructional delivery.
5. A system for tailoring feedback based on historical performance data to provide individualized coaching recommendations.

Further, in some embodiments, the present system evaluates individual sports performance across multiple modes, including scorecards, visual video overlays, and collaborative coaching sessions through WebRTC or similar real-time communication technologies. By leveraging AI and temporal data analysis, the system offers tailored insights, ensuring athletes receive precise, context-aware feedback and instructional support during and after their performance. The present disclosure relates to systems and methods for AI-powered performance evaluation and instruction in sports. Specifically, the disclosed system focuses on delivering real-time feedback, adaptive scoring, and personalized coaching through various feedback formats, such as scorecards, video overlays, and peer-to-peer communication technologies.

Further, in some embodiments, advances in sports analytics and AI have enabled automated systems to deliver adaptive feedback tailored to the specific needs of athletes. Moreover, the increasing use of collaborative technologies, such as WebRTC or similar frameworks, has opened the door for remote coaching, enabling athletes to access personalized feedback beyond physical spaces. The present disclosure combines multiple feedback channels with AI models trained on temporal data streams, ensuring athletes and coaches receive actionable insights in the moment. The incorporation of real-time communication frameworks also supports collaborative coaching sessions, improving athlete development and engagement.

Further, the present disclosure introduces a platform for real-time feedback and personalized instruction in sports performance. Key features include:
1. Multiple Feedback Formats—Real-time delivery through scorecards, video overlays, and live coaching via real-time communication technologies.
2. Adaptive AI Scoring Models—AI-driven models dynamically evaluate performance based on sport-specific mechanics and temporal data analysis.
3. Collaborative Coaching—Peer-to-peer coaching sessions supported through WebRTC or other real-time communication frameworks.
4. Seamless Feedback Synchronization—Synchronization of feedback with video streams and overlays for accurate instructional delivery.
5. Personalized Instruction - Feedback tailored to each athlete, based on historical performance data and adaptive scoring models.

Further, in some embodiments, the present disclosure may provide the following features:
1. Real-Time Feedback and Adaptive Instruction
   The disclosed system integrates various feedback formats—such as scorecards, visual overlays, and live coaching sessions—into a unified platform for sports performance evaluation. The platform continuously monitors an athlete's mechanics, such as shot phases (e.g., Transfer, Pocket, Release), and dynamically adjusts the feedback provided.
      Feedback is delivered via:
Scorecards: Summarizing key performance metrics for review.
Video Overlays: Displaying real-time annotations to highlight actions and areas for improvement.
Live Coaching Sessions: Real-time feedback via WebRTC or similar technologies to support remote coaching.
2. Collaborative Coaching through Real-Time Communication Technologies
   The disclosed system enables collaborative coaching by integrating WebRTC or similar real-time communication frameworks. Coaches and athletes may engage in interactive sessions, receiving and delivering personalized feedback remotely. Peer-to-peer sessions also allow athletes to collaborate with teammates in real-time, enhancing skill development and motivation.
3. Adaptive Scoring Models Based on Temporal Data
   At the core of the disclosed system are adaptive AI scoring models that analyze performance dynamically. The said models capture the temporal progression of actions (e.g., shot phases) and provide real-time evaluations based on:
      Historical Performance Data: Comparisons of past and current performance.
      Phase-Specific Metrics: Evaluations tailored to the mechanics of specific phases, such as Transfer, Pocket, or Release.
      Temporal Data Streams: Continuous data streams that track phase transitions for in-the-moment feedback.
4. Personalized Instruction and Feedback Delivery
   The system tailors feedback to each athlete's needs using their historical performance data and adaptive AI models. Key delivery methods include:
      Video Overlays: Annotated video streams displaying key performance insights.
      Scorecards: Detailed post-session reports summarizing performance.
      Live Feedback Streams: Real-time insights delivered during live sessions via WebRTC or similar communication technologies.

Further, in some embodiments, the present disclosure may have the following aspects:
1. System for Real-Time Feedback through Multiple Formats
   A system that provides real-time feedback using scorecards, video overlays, and personalized instruction via AI models.
2. Integration of Real-Time Communication Technologies for Collaborative Coaching
   A system that supports peer-to-peer coaching sessions through WebRTC or similar communication frameworks.
3. Adaptive Scoring Based on Temporal Data
   A method for generating personalized feedback using adaptive AI scoring models that analyze temporal data and track phase transitions.
4. Seamless Synchronization of Feedback with Video Streams
   A method for synchronizing AI-generated feedback with live or recorded video streams for enhanced instructional delivery.
5. Personalized Instruction Based on Athlete Data
   A system that delivers tailored feedback based on historical performance data, providing individualized coaching recommendations.
Further, the present disclosure introduces a comprehensive system for AI-driven, real-time feedback and instruction in sports performance. By integrating multiple feedback formats such as scorecards, video overlays, and collaborative coaching via real-time communication technologies, the system delivers timely, actionable insights. Adaptive AI scoring models evaluate mechanics dynamically using temporal data, while personalized instruction ensures that athletes receive tailored feedback. The given seamless integration of insights, streaming technologies, and collaborative coaching offers a powerful solution for improving sports performance across a variety of scenarios.

Further, the disclosed method and system may analyze a sports equipment (e.g., balls, hockey sticks, baseball bats, etc.) in addition to the biomechanical data for human motion. The sports equipment is as integral to athletic performance as the athlete's biomechanics, yet existing systems may not capture the same. For instance:
Golf: While some systems visualize body key points, they fail to track and represent the club and the club's interaction with the swing plane.
Hockey: The stick's position and interaction with the puck are critical for shot accuracy and passing mechanics.
Basketball: Tracking both the ball's trajectory and player motion allows for comprehensive analysis of dribbling, shooting, and passing mechanics.

Further, the present disclosure describes the following features associated with the disclosed system:
1. Equipment Integration in 3D Models:
   The disclosed system may incorporate sports equipment into 3D spatial analyses, ensuring that the equipment is represented as part of the biomechanical and situational evaluations. Quaternions or similar methods may track rotations and interactions between the athlete and equipment.
2. Composite Performance Metrics:
   The disclosed system may develop metrics that integrate athlete and equipment data (for example, the alignment between a player's hand and a basketball during release or the angle of a hockey stick blade during a slap shot).
3. Dynamic Equipment Modeling:
   The disclosed system may allow for adaptable modeling of different equipment types, enabling evaluations across multiple sports contexts.
4. Feedback and Instruction Enhancements:
   The disclosed system may provide real-time feedback that includes both the athlete and equipment (for example, suggesting grip adjustments or highlighting inconsistencies in equipment alignment).

In some embodiments, the present disclosure relates to the application of generative artificial intelligence (AI) for processing, analyzing, and visualizing integrated biomechanical datasets. The said datasets encompass human biomechanics, sports equipment dynamics, and associated visual and performance data.

Further, the emergence of integrated biomechanical datasets—combining 6D human movement data (spatial and rotational), sports equipment dynamics, and derived insights—presents both significant challenges and transformative potential. While advancements in pose estimation, video analysis, and AI-driven performance metrics have begun to address biomechanical analysis, the following challenges remain:
1. Data Volume and Complexity: The sheer scale and complexity of integrated biomechanical datasets make it difficult to extract actionable insights.
2. Role-Specific and Contextual Insights: Current systems lack the capability to tailor findings based on the needs of different stakeholders, such as coaches, athletes, and medical professionals.
3. Interactive Visualization: The dynamic interplay between human biomechanics and equipment dynamics requires advanced visualization techniques to highlight critical aspects of performance effectively.
4. Multi-Modal Interaction: Traditional systems are limited in their ability to offer multi-modal outputs, such as written reports, real-time visualizations, or conversational AI interfaces, to address diverse user preferences.

Further, the present disclosure describes a generative AI framework to overcome the said challenges, enabling robust analysis, summarization, and visualization of integrated biomechanical data in a user-specific and multi-modal manner.

Further, in some embodiments, the present disclosure may be understood using the following example:
Scenario: A basketball coach is reviewing a training session with a player.
   Coach: “Summarize the key areas where the player needs improvement during the shooting phase.”
   System Response: “During the transfer and release phases, the player's wrist rotation is inconsistent, deviating by 15 degrees from the ideal baseline. Additionally, the elbow extension is 10% slower than optimal, potentially affecting shot accuracy.”
   Coach: “Show me the 3D view of the transfer phase highlighting these deviations.”
   System Response: (Generates and displays a 3D visualization from an overhead angle, emphasizing wrist and elbow metrics with annotated deviations.)
   Coach: “compare This Session's Metrics to Last Week's.”
   System Response: “Compared to last week, wrist rotation consistency has improved by 8%, but elbow extension speed has declined by 5%. Would you like suggestions to improve the player's release timing?”
   Coach: “yes, Give me Recommendations.”
   System Response: “Focus on exercises targeting wrist stabilization and rapid elbow extension. Would you like me to generate a customized training plan?”

Further, the present disclosure leverages generative AI to distil complex integrated biomechanical datasets into actionable insights and dynamic visualizations. By incorporating human and equipment performance data, creating a holistic framework for real-time analysis, asynchronous review, and interactive feedback.

Further, in some embodiments, the present disclosure may provide the following benefits:
1. Role-Specific Summarization: Tailored insights for various stakeholders (e.g., coaches, athletes, analysts) based on their specific focus areas.
2. Dynamic Visualization and Perspective Control: AI-driven visualization tools that adjust views to highlight critical performance details, such as wrist motion during a basketball shot or stick alignment in hockey.
3. Interactive Q&A and Decision Support: Multi-modal generative AI interfaces enabling users to ask questions, explore insights, and receive adaptive feedback in real time or asynchronously.
4. Integration of External Knowledge Sources: The system may ingest supplementary materials, such as playbooks, medical reports, or regulations, enriching analytical capabilities and context-aware responses.
5. Multi-Agent Framework: Collaborative AI agents handle specific aspects of data analysis, visualization, and content generation, ensuring comprehensive coverage of the dataset.

Further, the generative AI system leverages multiple advanced technologies and proprietary frameworks to process and analyze integrated biomechanical datasets. Key components include:
1. Integrated Biomechanical Data Sources:
   The disclosed system ingests highly structured data from multiple sources, including:
      3D skeletal models with sports equipment integration (e.g., player's body and hockey stick).
      Rotational and positional data in 6D space.
      Heuristics and performance metrics defined through our proprietary frameworks.
2. Embedding and Vectorization:
   The disclosed system utilizes embedding engines to convert structured biomechanical data into vector representations:
      Knowledge Graphs: Representing activities, phases, and their hierarchical relationships for reasoning and exploration.
      Similarity Vectors: Used for player identification, activity type recognition, and heuristic comparisons.
3. Hybrid Graph Retrieval-Augmented Generation (Hybrid RAG):
   By combining knowledge graphs with traditional vector retrieval mechanisms, the system dynamically generates factual insights tailored to the query context.
4. Generative AI Models
   Incorporating vision and language models fine-tuned for sports biomechanics:
      Vision models provide spatial and temporal analysis.
      Language models generate detailed summaries and engage in domain-specific Q&A.
5. Multimodal Interfaces:
   The disclosed system supports interactive experiences via:
      Text-based chat for insights and detailed explanations.
      Audio responses for hands-free coaching interactions.
      Dynamic 3D visualizations, powered by integrated frameworks like Babylon.js and Unity, for enhanced understanding.
6. AI Optimization and Guardrails:
   Leveraging tools like NeMo Guardrails, the system ensures:
      Factual consistency in generated insights.
      Topic focus to maintain relevance during interactions.
7. Data Storage and Processing:
   Hybrid RAG Architecture: Combines graph databases, vector stores, and traditional relational databases for comprehensive data management.
   Kubernetes-Orchestrated Services: Ensuring scalable and robust performance for AI inference, data retrieval, and user interaction.
8. Multimodal Summarization and Visualization:
   The disclosed system produces interactive summaries and visual reports:
      Automatically generated 3D views highlighting key performance metrics.
      Interactive comparison tools for tracking player progress over time.

Further, in some embodiments, the disclosed system may provide the following benefits:
1. Generative AI for Integrated Biomechanical Data Analysis:
   A method for synthesizing insights from integrated biomechanical datasets, encompassing human biomechanics, equipment performance, and associated visual data, using generative AI.
2. Dynamic Visualization and Perspective Adjustment:
   A system that generates dynamic visualizations and selects optimal perspectives for highlighting key biomechanical features, incorporating both human and equipment dynamics.
3. Role-Specific Summarization and Feedback:
   A method for generating role-specific insights and feedback, tailored to the needs of diverse stakeholders, based on integrated biomechanical datasets.
4. Multi-Modal Output and Interaction:
   A system supporting interactive outputs in text, audio, visual, and conversational formats, enabling multi-modal engagement with generative AI insights.
5. Integration of External Knowledge Sources:
   A method for enhancing generative AI analysis by integrating external documents and datasets, such as training guidelines or medical records, into the decision-making process.
6. Multi-Agent Collaboration for Biomechanical Insights:
   A framework allowing multiple AI agents to collaboratively analyze integrated biomechanical datasets, manage content streams, and generate insights dynamically.

Further, the present disclosure tackles the challenges of scaling and contextualizing integrated biomechanical data analysis by combining human biomechanics and equipment performance into unified datasets, enabling comprehensive analysis and dynamic feedback.

Further, the generative AI framework processes integrated datasets to extract meaningful insights, including:
Identifying inefficiencies in shooting mechanics.
Highlighting improvements over time in rehabilitation scenarios.
Generating tailored summaries for specific user roles, such as shooting coaches or rehabilitation therapists.

Further, the system leverages AI to dynamically adjust perspectives in 3D/6D visualizations, such as:
Automatically rotating the view to highlight wrist mechanics during a basketball shot.
Generating overlays to compare actual performance against saved baselines, including both body mechanics and equipment alignment.

Further, users may upload supplementary documents (e.g., playbooks or injury reports), enabling the system to provide enriched, context-aware insights.

Further, the collaborative AI agents manage tasks such as:
Contextualizing integrated biomechanical datasets.
Generating real-time or asynchronous insights.
Handling user queries and dynamically adjusting visualizations or summaries.

Further, the present disclosure represents a paradigm shift in integrated biomechanical data analysis by leveraging generative AI to provide actionable insights and dynamic visualizations. By addressing key challenges such as data volume, visualization limitations, and user-specific needs, the disclosed system empowers users to unlock the full potential of integrated biomechanical datasets, revolutionizing sports performance, rehabilitation, and training.

In some embodiments, the present disclosure relates to the field of sports performance analysis and coaching, particularly focusing on real-time 3D skeletal tracking, visualization, and collaborative coaching. Further, the present disclosure provides an interactive platform where coaches and athletes may make real-time adjustments to 3D skeletons. The system also enables synchronization of the said adjustments across sessions through distributed data platforms and facilitates both live and asynchronous coaching with session recording and review capabilities. Additionally, current solutions typically do not support synchronized session data across distributed platforms or offer collaborative coaching tools with asynchronous review options.

Further, the disclosed system introduces a real-time 3D skeleton adjustment and collaborative coaching platform designed for dynamic interaction between coaches and athletes. The system leverages distributed messaging systems like Redis and Kafka to synchronize changes across sessions and ensure consistency. Further, the disclosed system supports live interactions using WebRTC or similar technologies and offers session recordings for asynchronous review and feedback.

Further, in some embodiments, the present disclosure enables real-time adjustments to 3D integrated biomechanical models, which are rendered using frameworks such as Babylon.js. The said adjustments may be shared across multiple coaching sessions and participants in real time, with performance data and changes stored for later analysis. Coaches may use visual overlays and collaborative tools to provide precise feedback, enhancing the athlete's learning and improvement.

Further, in some embodiments, the present disclosure may describe the following benefits associated with the disclosed system:
1. Real-time 3D Skeleton Adjustment System:
   A system that allows real-time adjustments to 3D skeletons, rendered through visualization frameworks, for precise performance analysis.
2. Collaborative Coaching With Live Synchronization:
   A collaborative coaching platform that synchronizes adjustments and session data across multiple participants and devices using Redis, Kafka, or similar technologies.
3. WebRTC or Similar Technology Integration:
   Integration of WebRTC or comparable peer-to-peer communication technologies to facilitate real-time collaboration and coaching.
4. Asynchronous Review through Session Recordings:
   A method for recording and archiving collaborative coaching sessions, allowing asynchronous review and performance feedback.
5. Multi-participant Session Management:
A method for managing multiple participants in live or recorded coaching sessions, enabling seamless interaction across distributed locations.

Further, in some embodiments, the present disclosure may describe the following aspects associated with the disclosed system:
1. Real-Time 3D Skeleton Adjustment System
   The system renders 3D skeletons of athletes in real time using frameworks like Babylon.js or equivalent technologies. Coaches may adjust joint positions, angles, and movement paths directly within the rendered skeleton during live sessions. The said adjustments are reflected immediately across all connected participants to provide synchronized feedback.
2. Collaborative Coaching and Synchronization
   The system facilitates collaborative coaching through the use of WebRTC or other peer-to-peer communication technologies, enabling real-time interaction between athletes and coaches. Session data and adjustments are synchronized using Redis/Kafka platforms to ensure consistency across devices and participants. For example, during a basketball coaching session, if a coach adjusts an athlete's shooting arm position in the 3D skeleton, the change is reflected instantly on all devices connected to the session, ensuring that both the coach and athlete are working with the same visual representation of the movement.
3. Session Recording and Asynchronous Review
   All collaborative sessions may be recorded and archived for asynchronous review. Coaches may provide additional feedback post-session, and athletes may revisit the said sessions to analyze their performance. The given capability ensures that learning is

continuous and not limited to live interactions.
4. Multi-Participant Sessions and Distributed Management
   The system supports multiple participants across distributed locations. Coaches, athletes, and other stakeholders may join live or recorded sessions, providing a flexible environment for individual and team coaching. Redis or Kafka platforms manage participant data and synchronization, ensuring seamless session continuity.

Further, in some embodiments, the present disclosure introduces a real-time, interactive 3D skeleton adjustment and collaborative coaching system. Leveraging technologies such as WebRTC, Redis, and Kafka, the platform enables precise performance analysis, synchronized coaching sessions, and continuous feedback through live and recorded interactions. It addresses the challenges of remote and asynchronous coaching, providing athletes and coaches with a comprehensive and adaptive tool for improving sports performance across various scenarios.

Further, in some embodiments, the present disclosure describes a real-time skeleton adjustment capability through collaborative tools powered by WebRTC or similar technologies, enabling coaches and athletes to interactively adjust 3D skeletons rendered using frameworks like Babylon.js or equivalent tools, ensuring synchronized changes across sessions. The system leverages Redis, Kafka, or similar platforms to manage synchronization and data consistency, allowing seamless collaboration. Sessions may be recorded for asynchronous review, facilitating continuous learning and performance improvement.

Further, in some embodiments, the present disclosure describes:
1. A system that enables real-time adjustments of 3D skeletons with collaborative tools for interactive performance analysis.
2. Synchronization of changes across multiple coaching sessions using Redis, Kafka, or similar distributed messaging platforms.
3. Integration of WebRTC or comparable peer-to-peer communication technologies to support real-time collaborative coaching.
4. A method for recording collaborative coaching sessions, enabling asynchronous review and feedback for continuous performance improvement.

Support for multi-participant sessions with seamless data management and synchronization across distributed locations.

The present disclosure provides a method of facilitating real-time performance feedback as a software-as-a-service. Further, the method may include receiving, using a communication device, a video stream data representing an athletic performance from a client device. Further, the method may include receiving, using the communication device, a historical performance data associated with a user from the client device. Further, the method may include analyzing, using a processing device, the video stream data as temporal data to identify a performance phase. Further, the method may include determining, using the processing device, an adaptive performance score based on the performance phase and the historical performance data. Further, the method may include generating, using the processing device, a synchronized feedback data aligned with the video stream data based on the adaptive performance score. Further, the method may include storing, using a storage device, the synchronized feedback data. Further, the method may include transmitting, using the communication device, the synchronized feedback data to the client device.

Further, in some embodiments, the analyzing the video stream data may include extracting, using the processing device, a temporal feature data from the video stream data. Further, the analyzing the video stream data may include identifying, using the processing device, a transition point within the temporal feature data. Further, the analyzing the video stream data may include assigning, using the processing device, a defined temporal phase label to the video stream data.

Further, in some embodiments, the determining the adaptive performance score may include retrieving, using the storage device, a reference performance data associated with the historical performance data. Further, the determining the adaptive performance score may include comparing, using the processing device, the reference performance data with the video stream data. Further, the determining the adaptive performance score may include weighting, using the processing device, the adaptive performance score based on a deviation between the reference performance data and the video stream data.

Further, in some embodiments, the method includes aggregating, using the processing device, two or more adaptive performance score values into a summary metric. Further, in some embodiments, the method includes generating, using the processing device, a scorecard data based on the summary metric.

Further, in some embodiments, the generating the synchronized feedback data may include calculating, using the processing device, a spatial alignment parameter corresponding to the video stream data. Further, the generating the synchronized feedback data may include generating, using the processing device, an overlay annotation data based on the adaptive performance score. Further, the generating the synchronized feedback data may include temporally aligning, using the processing device, the overlay annotation data with the video stream data.

Further, in some embodiments, the method includes packaging, using the processing device, the synchronized feedback data into a real-time transmission format. Further, in some embodiments, the method includes transmitting, using the communication device, the synchronized feedback data during an active real-time communication session.

Further, in some embodiments, the determining the adaptive performance score may include analyzing, using the processing device, the historical performance data to identify a performance trend data. Further, the determining the adaptive performance score may include computing, using the processing device, a personalization factor based on the performance trend data. Further, the determining the adaptive performance score may include applying, using the processing device, the personalization factor to the adaptive performance score.

Further, in some embodiments, the generating the synchronized feedback data may include detecting, using the processing device, a change event in the performance phase. Further, the generating the synchronized feedback data may include recalculating, using the processing device, the adaptive performance score in response to the change event. Further, the generating the synchronized feedback data may include updating, using the processing device, the synchronized feedback data based on the recalculated adaptive performance score.

Further, in some embodiments, the method includes associating, using the processing device, the synchronized feedback data with a corresponding timestamp of the video stream data. Further, in some embodiments, the method includes storing, using the storage device, the synchronized feedback data with the associated timestamp.

Further, in some embodiments, the analyzing the video stream data may include retrieving, using the storage device, a previously stored synchronized feedback data. Further, the analyzing the video stream data may include extracting, using the processing device, a feedback pattern data from the previously stored synchronized feedback data. Further, the analyzing the video stream data may include incorporating, using the processing device, the feedback pattern data into a current analysis of the video stream data.

The present disclosure provides a system for facilitating real-time performance feedback as a software as a service. Further, the system may include a communication device. Further, the communication device may be configured for receiving a video stream data representing an athletic performance from a client device. Further, the communication device may be configured for receiving a historical performance data associated with a user from the client device. Further, the communication device may be configured for transmitting a synchronized feedback data to the client device. Further, the system may include a processing device. Further, the processing device may be configured for analyzing the video stream data as temporal data to identify a performance phase. Further, the processing device may be configured for determining an adaptive performance score based on the performance phase and the historical performance data. Further, the processing device may be configured for generating the synchronized feedback data aligned with the video stream data based on the adaptive performance score. Further, the system may include a storage device which may be configured for storing the synchronized feedback data.

Further, in some embodiments, the processing device may be further configured for extracting a temporal feature data from the video stream data. Further, the processing device may be further configured for identifying a transition point within the temporal feature data. Further, the processing device may be further configured for assigning a defined temporal phase label to the video stream data.

Further, in some embodiments, the storage device may be further configured for retrieving a reference performance data associated with the historical performance data. Further, the processing device may be further configured for comparing the reference performance data with the video stream data. Further, the processing device may be further configured for weighting the adaptive performance score based on a deviation between the reference performance data and the video stream data.

Further, in some embodiments, the processing device may be further configured for aggregating two or more adaptive performance score values into a summary metric. Further, the processing device may be further configured for generating a scorecard data based on the summary metric.

Further, in some embodiments, the processing device may be further configured for calculating a spatial alignment parameter corresponding to the video stream data. Further, the processing device may be further configured for generating an overlay annotation data based on the adaptive performance score. Further, the processing device may be further configured for temporally aligning the overlay annotation data with the video stream data.

In some embodiments, the processing device may be further configured for packaging the synchronized feedback data into a real-time transmission format. Further, the communication device may be further configured for transmitting the synchronized feedback data during an active real-time communication session.

Further, in some embodiments, the processing device may be further configured for analyzing the historical performance data to identify a performance trend data. Further, the processing device may be further configured for computing a personalization factor based on the performance trend data. Further, the processing device may be further configured for applying the personalization factor to the adaptive performance score.

Further, in some embodiments, the processing device may be further configured for detecting a change event in the performance phase. Further, the processing device may be further configured for recalculating the adaptive performance score in response to the change event. Further, the processing device may be further configured for updating the synchronized feedback data based on the recalculated adaptive performance score.

In some embodiments, the processing device may be further configured for associating the synchronized feedback data with a corresponding timestamp of the video stream data. Further, the storage device may be further configured for storing the synchronized feedback data with the associated timestamp.

Further, in some embodiments, the storage device may be further configured for retrieving a previously stored synchronized feedback data. Further, the processing device may be further configured for extracting a feedback pattern data from the previously stored synchronized feedback data. Further, the processing device may be further configured for incorporating the feedback pattern data into a current analysis of the video stream data.

The present disclosure provides a method of facilitating real-time personalized sports performance feedback. Further, the method may include receiving, using a communication device, an integrated biomechanical data from a motion capture system, the integrated biomechanical data comprising a human motion data and an equipment dynamic data. Further, the method may include analyzing, using a processing device, the integrated biomechanical data to determine a composite performance metric representing a combined relationship between the human motion data and the equipment dynamic data. Further, the method may include generating, using the processing device, a feedback data based on the composite performance metric. Further, the method may include storing, using a storage device, the feedback data. Further, the method may include transmitting, using the communication device, the feedback data to a client device.

Further, in some embodiments, the method includes determining, using the processing device, a user role data associated with the client device. Further, in some embodiments, the method includes adapting, using the processing device, the feedback data based on the user role data.

Further, in some embodiments, the method includes analyzing, using the processing device, the integrated biomechanical data across a time interval. Further, in some embodiments, the method includes identifying, using the processing device, a temporal deviation data representing variation from a baseline state.

Further, in some embodiments, the determining the composite performance metric may include calculating, using the processing device, a spatial relationship data between the human motion data and the equipment dynamic data. Further, the determining the composite performance metric may include combining, using the processing device, the spatial relationship data into the composite performance metric.

Further, in some embodiments, the method includes applying, using the processing device, an adaptive scoring model to the integrated biomechanical data. Further, in some embodiments, the method includes updating, using the processing device, the composite performance metric based on the adaptive scoring model.

Further, in some embodiments, the method includes determining, using the processing device, a visualization perspective data associated with a biomechanical deviation. Further, in some embodiments, the method includes generating, using the processing device, a visualization data based on the visualization perspective data.

Further, in some embodiments, the method includes receiving, using the communication device, a query data associated with the feedback data. Further, in some embodiments, the method includes generating, using the processing device, a response data based on the query data and the integrated biomechanical data.

Further, in some embodiments, the method includes retrieving, using the storage device, a historical biomechanical data associated with the client device. Further, in some embodiments, the method includes comparing, using the processing device, the integrated biomechanical data with the historical biomechanical data.

Further, in some embodiments, the generating the feedback data may include analyzing, using the processing device, an equipment alignment data derived from the equipment dynamic data. Further, the generating the feedback data may include annotating, using the processing device, the feedback data based on the equipment alignment data.

Further, in some embodiments, the method includes structuring, using the processing device, the feedback data into a multi-modal output data. Further, in some embodiments, the method includes transmitting, using the communication device, the multi-modal output data to the client device.

The present disclosure provides a system for facilitating real-time personalized sports performance feedback. Further, the system may include a communication device. Further, the communication device may be configured for receiving an integrated biomechanical data from a motion capture system, the integrated biomechanical data comprising a human motion data and an equipment dynamic data. Further, the communication device may be configured for transmitting a feedback data to a client device. Further, the system may include a processing device. Further, the processing device may be configured for analyzing the integrated biomechanical data to determine a composite performance metric representing a combined relationship between the human motion data and the equipment dynamic data. Further, the processing device may be configured for generating the feedback data based on the composite performance metric. Further, the system may include a storage device which may be configured for storing the feedback data.

Further, in some embodiments, the processing device may be further configured for determining a user role data associated with the client device. Further, the processing device may be further configured for adapting the feedback data based on the user role data.

Further, in some embodiments, the processing device may be further configured for analyzing the integrated biomechanical data across a time interval. Further, the processing device may be further configured for identifying a temporal deviation data representing variation from a baseline state.

Further, in some embodiments, the processing device may be further configured for calculating a spatial relationship data between the human motion data and the equipment dynamic data. Further, the processing device may be further configured for combining the spatial relationship data into the composite performance metric.

Further, in some embodiments, the processing device may be further configured for applying an adaptive scoring model to the integrated biomechanical data. Further, the processing device may be further configured for updating the composite performance metric based on the adaptive scoring model.

Further, in some embodiments, the processing device may be further configured for determining a visualization perspective data associated with a biomechanical deviation. Further, the processing device may be further configured for generating a visualization data based on the visualization perspective data.

In some embodiments, the communication device may be further configured for receiving a query data associated with the feedback data. Further, the processing device may be further configured for generating a response data based on the query data and the integrated biomechanical data.

In some embodiments, the storage device may be further configured for retrieving a historical biomechanical data associated with the client device. Further, the processing device may be further configured for comparing the integrated biomechanical data with the historical biomechanical data.

Further, in some embodiments, the processing device may be further configured for analyzing an equipment alignment data derived from the equipment dynamic data. Further, the processing device may be further configured for annotating the feedback data based on the equipment alignment data.

In some embodiments, the processing device may be further configured for structuring the feedback data into a multi-modal output data. Further, the communication device may be further configured for transmitting the multi-modal output data to the client device.

The present disclosure provides a method of facilitating real-time collaborative adjustment of a three-dimensional skeleton for sports coaching. Further, the method may include receiving, using a communication device, skeleton data representing a three-dimensional skeleton from a client device. Further, the method may include receiving, using the communication device, adjustment data representing a modification to the three-dimensional skeleton from the client device. Further, the method may include determining, using a processing device, an updated skeleton data based on the adjustment data and the skeleton data. Further, the method may include storing, using a storage device, the updated skeleton data. Further, the method may include transmitting, using the communication device, the updated skeleton data to the client device.

Further, in some embodiments, the method includes synchronizing, using the processing device, the updated skeleton data with a session state data. Further, in some embodiments, the method includes storing, using the storage device, the session state data.

Further, in some embodiments, the method includes identifying, using the processing device, a collaboration state associated with the updated skeleton data. Further, in some embodiments, the method includes storing, using the storage device, the collaboration state.

Further, in some embodiments, the method includes verifying, using the processing device, consistency of the updated skeleton data with a prior session data. Further, in some embodiments, the method includes storing, using the storage device, the prior session data.

Further, in some embodiments, the method includes transmitting, using the communication device, a synchronization signal associated with the updated skeleton data. Further, in some embodiments, the method includes storing, using the storage device, the synchronization signal.

Further, in some embodiments, the method includes recording, using the processing device, a session record based on the updated skeleton data. Further, in some embodiments, the method includes storing, using the storage device, the session record.

Further, in some embodiments, the method includes retrieving, using the storage device, the session record. Further, in some embodiments, the method includes transmitting, using the communication device, the session record to the client device.

Further, in some embodiments, the method includes associating, using the processing device, a participant data with the updated skeleton data. Further, in some embodiments, the method includes storing, using the storage device, the participant data.

Further, in some embodiments, the method includes generating, using the processing device, a biomechanical parameter based on the updated skeleton data. Further, in some embodiments, the method includes storing, using the storage device, the biomechanical parameter.

Further, in some embodiments, the method includes analyzing, using the processing device, a sequence of updated skeleton data over time. Further, in some embodiments, the method includes storing, using the storage device, a performance feedback data derived from the analysis.

The present disclosure provides a system for facilitating real-time collaborative adjustment of a three-dimensional skeleton for sports coaching. Further, the system may include a communication device. Further, the communication device may be configured for receiving skeleton data representing a three-dimensional skeleton from a client device. Further, the communication device may be configured for receiving adjustment data representing a modification to the three-dimensional skeleton from the client device. Further, the communication device may be configured for transmitting updated skeleton data to the client device. Further, the system may include a processing device which may be configured for determining an updated skeleton data based on the adjustment data and the skeleton data. Further, the system may include a storage device which may be configured for storing the updated skeleton data.

In some embodiments, the processing device may be configured for synchronizing the updated skeleton data with a session state data. Further, the storage device may be configured for storing the session state data.

In some embodiments, the processing device may be further configured for identifying a collaboration state associated with the updated skeleton data. Further, the storage device may be configured for storing the collaboration state.

In some embodiments, the processing device may be further configured for verifying consistency of the updated skeleton data with a prior session data. Further, the storage device may be further configured for storing the prior session data.

In some embodiments, the communication device may be further configured for transmitting a synchronization signal associated with the updated skeleton data. Further, the storage device may be further configured for storing the synchronization signal.

In some embodiments, the processing device may be further configured for recording a session record based on the updated skeleton data. Further, the storage device may be further configured for storing the session record.

In some embodiments, the storage device may be further configured for retrieving the session record. Further, the communication device may be further configured for transmitting the session record to the client device.

In some embodiments, the processing device may be further configured for associating a participant data with the updated skeleton data. Further, the storage device may be further configured for storing the participant data.

In some embodiments, the processing device may be further configured for generating a biomechanical parameter based on the updated skeleton data. Further, the storage device may be further configured for storing the biomechanical parameter.

In some embodiments, the processing device may be further configured for analyzing a sequence of updated skeleton data over time. Further, the storage device may be further configured for storing a performance feedback data derived from the analysis.

Further, in some embodiments, the method includes determining, using the processing device, a user role data associated with the client device. Further, in some embodiments, the method includes adapting, using the processing device, the feedback data based on the user role data.

Further, in some embodiments, the method includes analyzing, using the processing device, the integrated biomechanical data across a time interval. Further, in some embodiments, the method includes identifying, using the processing device, a temporal deviation data representing variation from a baseline state.

Further, in some embodiments, the determining the composite performance metric includes calculating, using the processing device, a spatial relationship data between the human motion data and the equipment dynamic data. Further, the determining the composite performance metric may include combining, using the processing device, the spatial relationship data into the composite performance metric.

Further, in some embodiments, the method includes applying, using the processing device, an adaptive scoring model to the integrated biomechanical data. Further, in some embodiments, the method includes updating, using the processing device, the composite performance metric based on the adaptive scoring model.

Further, in some embodiments, the method includes determining, using the processing device, a visualization perspective data associated with a biomechanical deviation. Further, in some embodiments, the method includes generating, using the processing device, a visualization data based on the visualization perspective data.

Further, in some embodiments, the method includes receiving, using the communication device, a query data associated with the feedback data. Further, in some embodiments, the method includes generating, using the processing device, a response data based on the query data and the integrated biomechanical data.

Further, in some embodiments, the method includes retrieving, using the storage device, a historical biomechanical data associated with the client device. Further, in some embodiments, the method includes comparing, using the processing device, the integrated biomechanical data with the historical biomechanical data.

Further, in some embodiments, the generating the feedback data may include analyzing, using the processing device, an equipment alignment data derived from the equipment dynamic data. Further, the generating the feedback data may include annotating, using the processing device, the feedback data based on the equipment alignment data.

Further, in some embodiments, the method includes structuring, using the processing device, the feedback data into a multi-modal output data. Further, in some embodiments, the method includes transmitting, using the communication device, the multi-modal output data to the client device.

The present disclosure provides a system for facilitating real-time personalized sports performance feedback. Further, the system may include a communication device. Further, the communication device may be configured for receiving an integrated biomechanical data from a motion capture system, the integrated biomechanical data comprising a human motion data and an equipment dynamic data. Further, the communication device may be configured for transmitting a feedback data to a client device. Further, the system may include a processing device. Further, the processing device may be configured for analyzing the integrated biomechanical data to determine a composite performance metric representing a combined relationship between the human motion data and the equipment dynamic data. Further, the processing device may be configured for generating the feedback data based on the composite performance metric. Further, the system may include a storage device which may be configured for storing the feedback data.

Further, in some embodiments, the processing device may be further configured for determining a user role data associated with the client device. Further, the processing device may be further configured for adapting the feedback data based on the user role data.

Further, in some embodiments, the processing device may be further configured for analyzing the integrated biomechanical data across a time interval. Further, the processing device may be further configured for identifying a temporal deviation data representing variation from a baseline state.

Further, in some embodiments, the processing device may be further configured for calculating a spatial relationship data between the human motion data and the equipment dynamic data. Further, the processing device may be further configured for combining the spatial relationship data into the composite performance metric.

Further, in some embodiments, the processing device may be further configured for applying an adaptive scoring model to the integrated biomechanical data. Further, the processing device may be further configured for updating the composite performance metric based on the adaptive scoring model.

Further, in some embodiments, the processing device may be further configured for determining a visualization perspective data associated with a biomechanical deviation. Further, the processing device may be further configured for generating a visualization data based on the visualization perspective data.

In some embodiments, the communication device may be further configured for receiving a query data associated with the feedback data. Further, the processing device may be further configured for generating a response data based on the query data and the integrated biomechanical data.

In some embodiments, the storage device may be further configured for retrieving a historical biomechanical data associated with the client device. Further, the processing device may be further configured for comparing the integrated biomechanical data with the historical biomechanical data.

Further, in some embodiments, the processing device may be further configured for analyzing an equipment alignment data derived from the equipment dynamic data. Further, the processing device may be further configured for annotating the feedback data based on the equipment alignment data.

In some embodiments, the processing device may be further configured for structuring the feedback data into a multi-modal output data. Further, the communication device may be further configured for transmitting the multi-modal output data to the client device.

In some embodiments, the disclosed system may inherently improve the technical field of real-time biomechanical data processing by addressing the technical problem that conventional sports analytics systems are unable to reliably analyze temporally evolving biomechanical and equipment-interaction data streams at low latency while preserving contextual accuracy. In some embodiments, the disclosed system may implement adaptive temporal phase-aware inference pipelines in which biomechanical motion is segmented into dynamically learned micro-phases rather than static, pre-defined phases. Such micro-phases may be derived using temporal convolutional networks, transformer-based sequence encoders, or hybrid recurrent-attention architectures that continuously re-segment motion based on observed kinematic discontinuities. In some embodiments, the given implementation may allow the system to detect subtle deviations in joint sequencing, equipment orientation, or force transfer that are not observable in frame-based or phase-agnostic systems, thereby improving the underlying technology of temporal motion analysis and real-time sports biomechanics inference.

In some embodiments, the disclosed system may inherently improve the technology of synchronized multimodal data fusion by solving the technical problem of temporal misalignment between video streams, biomechanical skeletal data, and equipment motion signals. In some embodiments, the system may implement clock-drift-corrected temporal anchoring mechanisms in which biomechanical events are indexed to invariant biomechanical landmarks rather than video frame timestamps alone. In some embodiments, such anchoring may be achieved using learned event-detection models that identify biomechanical inflection points, such as peak angular velocity or moment-of-inertia shifts, and use those points as synchronization anchors across modalities. In some embodiments, the given approach may be implemented using graph-based temporal alignment layers or differentiable time-warping models, thereby improving the technology of real-time multimodal synchronization and reducing instructional latency in live feedback systems.

In some embodiments, the disclosed system may inherently improve the field of integrated human-equipment biomechanical modeling by addressing the technical limitation that existing systems treat sports equipment as passive or external objects. In some embodiments, the disclosed system may model sports equipment as dynamically coupled rigid or semi-rigid bodies within a unified biomechanical graph, where joints, grip points, and contact surfaces are represented as constraint nodes. In some embodiments, such modeling may use quaternion-based rotational coupling, screw-theory representations, or learned constraint solvers that infer force transmission between the athlete and equipment. In some embodiments, the given implementation may enable the system to compute composite performance metrics that reflect coupled human-equipment dynamics, thereby improving the underlying technology of biomechanical simulation and performance evaluation.

In some embodiments, the disclosed system may inherently improve the technology of real-time instructional visualization by overcoming the problem that static or manually selected viewpoints fail to reveal critical biomechanical deviations. In some embodiments, the system may implement AI-driven viewpoint optimization engines that dynamically select camera perspectives, projection planes, or 3D render orientations based on detected biomechanical anomalies. In some embodiments, such engines may use reinforcement learning, saliency modeling, or information-gain maximization to determine which spatial perspective best exposes a deviation in joint alignment, equipment angle, or motion timing. In some embodiments, improving the technology of 3D visualization and instructional rendering by automating perspective control in response to live biomechanical data.

In some embodiments, the disclosed system may inherently improve the technical field of adaptive scoring systems by addressing the problem that conventional scoring models rely on static thresholds or population averages. In some embodiments, the system may implement self-normalizing, athlete-specific scoring models that continuously recalibrate performance baselines using Bayesian updating, continual learning, or personalized embedding spaces. In some embodiments, the system may generate individualized performance manifolds against which current actions are evaluated, thereby improving the technology of performance scoring by reducing bias introduced by generic reference models and enabling fine-grained personalization.

In some embodiments, the disclosed system may inherently improve the technology of real-time collaborative coaching by addressing the technical problem of state inconsistency across distributed participants. In some embodiments, the system may implement deterministic state replication for biomechanical models using event-sourced synchronization protocols layered on top of distributed messaging systems. In some embodiments, skeletal adjustments, annotations, and equipment transformations may be encoded as idempotent state-change events that are replayable and reversible across sessions. In some embodiments, improving the technology of distributed real-time collaboration by ensuring biomechanical state consistency across heterogeneous devices and network conditions.

In some embodiments, additional technical improvements may be incorporated to further enhance the underlying technologies used in the disclosed system. In some embodiments, the system may add uncertainty-aware biomechanical inference layers to address the technical problem that pose estimation and equipment tracking are inherently noisy. In some embodiments, probabilistic graphical models, ensemble predictors, or Bayesian neural networks may be used to propagate confidence intervals alongside biomechanical measurements. In some embodiments, the given feature may improve the technology of AI-based motion analysis by enabling feedback that is conditioned on measurement reliability rather than absolute values.

In some embodiments, the system may include federated or privacy-preserving model adaptation mechanisms to address the technical problem that centralized training on sensitive biomechanical data raises privacy and compliance concerns. In some embodiments, athlete-specific models may be updated locally using on-device learning and periodically aggregated using secure parameter-sharing protocols. In some embodiments, improving the technology of distributed machine learning in sports analytics by enabling personalization without centralized raw-data aggregation.

In some embodiments, the system may incorporate causal inference layers to address the technical problem that correlation-based feedback may not reliably identify root causes of performance issues. In some embodiments, the system may construct causal graphs linking joint kinematics, equipment dynamics, and outcome metrics, and may simulate counterfactual scenarios such as altered joint timing or grip orientation. In some embodiments, improving the technology of decision-support systems by enabling causally grounded coaching recommendations.

In some embodiments, the system may add cross-session longitudinal drift detection to address the technical problem that gradual performance degradation is difficult to detect using session-isolated analysis. In some embodiments, the system may implement temporal drift models that track slow changes in biomechanics over weeks or months using trend-aware embeddings or temporal clustering. In some embodiments, improving the technology of long-term performance monitoring and rehabilitation analytics.

In some embodiments, the system may integrate generative simulation engines to address the technical problem that athletes and coaches may not easily visualize hypothetical technique changes. In some embodiments, generative models may synthesize plausible future motion trajectories conditioned on modified biomechanical parameters, equipment adjustments, or training interventions. In some embodiments, improving the technology of instructional simulation by enabling predictive visualization rather than retrospective analysis.

Collectively, in some embodiments, the technical improvements described herein may transform the underlying technologies of real-time biomechanical analysis, multimodal synchronization, collaborative coaching, and AI-driven instructional systems by enabling context-aware, temporally precise, and personalized performance evaluation that is not achievable using conventional or routine techniques.

Further, the present disclosure describes a method of provisioning a feedback based on an activity. Further, in some embodiments, the method may include obtaining, using the processing device, activity data associated with at least one prior performance of the activity by the user, wherein the activity data includes time stamps and at least one sensor modality. Further, the method may include analyzing, using the processing device, the activity data associated with the at least one prior performance to determine at least one baseline parameter for the user by computing summary statistics over at least one time window. Further, in some embodiments, the at least one baseline parameter may include a baseline cadence, baseline peak velocity, baseline reaction time, baseline trajectory envelope, baseline pose range-of-motion, baseline accuracy metric, or combinations thereof. Further, the method may include configuring, using the processing device, the AI module based on the at least one baseline parameter, wherein configuring includes selecting one of a plurality of inference profiles stored in memory and associating the selected inference profile with the user. Further, in some embodiments, an inference profile includes (i) a feature subset definition, (ii) an inference sampling rate, (iii) a confidence threshold, and (iv) a message update interval. Further, in some embodiments, the confidence threshold is selected based on the baseline parameter such that false alerts are reduced for the particular user. Further, the generating of the feedback data may be performed using the configured AI module such that the feedback data is generated and provided within a predetermined end-to-end latency associated with the real-time communication protocol. Further, in some embodiments, the processing device enforces the predetermined end-to-end latency by (i) limiting inference to a maximum window length, (ii) limiting the number of features transmitted per update, and/or (iii) selecting a lower-complexity inference profile when measured delay exceeds the latency threshold.

Further, in some embodiments, the method may include analyzing, using the processing device, the activity data using a machine learning model configured for object detection and classification to identify a plurality of objects represented in the activity data. Further, in some embodiments, the activity data includes at least image frames, and the processing device generates an input tensor by resizing frames to a predetermined resolution, normalizing pixel values, and arranging channels in a predetermined order. Further, the method may include classifying, using the processing device, the plurality of objects into at least one of an individual and a sporting object. Further, in some embodiments, the machine learning model produces for each detected object an object indicator including (i) a class label, (ii) a confidence score, and (iii) a location descriptor, and the processing device selects an “individual” instance and a “sporting object” instance based on a rule stored in memory (e.g., highest confidence per class, temporal consistency across frames, or size/shape constraints). Further, the method may include isolating, using the processing device, individual activity data associated with activity of the individual and object activity data associated with activity of the sporting object from the activity data based on the classifying. Further, in some embodiments, isolating includes generating separate feature streams for the individual and the sporting object, where each feature stream comprises time-stamped location descriptors and confidence scores corresponding to the individual and the sporting object, respectively. Further, the generating of the feedback data may be based on at least one of the individual activity data or the object activity data, such that feedback reflects interaction between the individual and the sporting object rather than generic analysis of raw frames.

Further, in some embodiments, the method may include generating, using the processing device, a reduced feature representation from the activity data, wherein the reduced feature representation includes a predetermined set of feature fields stored in memory as a schema. Further, in some embodiments, the schema includes at least: (i) a time stamp, (ii) one or more kinematic features (position, velocity, acceleration), (iii) one or more interaction features (distance between entities, relative velocity, timing offsets), and (iv) a confidence value. Further, the reduced feature representation may exclude at least a portion of raw sensor samples by omitting raw pixel arrays and transmitting only derived features. Further, the method may include transmitting, using the communication device, the reduced feature representation to the user device using the real-time communication protocol in periodic updates having a stored update interval. Further, in some embodiments, updates are encoded as fixed-length or bounded-length messages, thereby enabling predictable packetization behavior and improved real-time transport. Further, the method may include generating, using the processing device, the feedback data based on the reduced feature representation. Further, transmitting the reduced feature representation may reduce bandwidth consumption by reducing at least one of (i) payload size per update, (ii) number of updates per second, or (iii) total bitrate, relative to transmitting raw sensor samples. Further, in some embodiments, the processing device verifies the bandwidth reduction by estimating bitrate using measured message sizes and update intervals and selecting a smaller feature subset when a bandwidth limit is approached.

Further, in some embodiments, the method may include extracting, using the processing device, a predetermined number of individual activity features from the individual activity data, wherein the predetermined number is a stored configuration value, and wherein the features represent activity of the individual. Further, the method may include extracting, using the processing device, a predetermined number of object activity features from the object activity data, wherein the predetermined number is a stored configuration value, and wherein the features represent activity of the sporting object. Further, in some embodiments, the individual activity features include at least one of: pose-derived joint angles, stride frequency, limb angular velocity, torso orientation, center-of-mass proxy location, or movement smoothness. Further, in some embodiments, the object activity features include at least one of: object speed, trajectory curvature, acceleration, bounce count, spin proxy, or release angle proxy. Further, the method may include correlating, using the processing device, the individual activity features with the object activity features by time-aligning features to a common time base. Further, in some embodiments, time-aligning includes resampling each feature stream to a common sampling interval and filling missing samples via interpolation or hold-last-sample. Further, the method may include forming, using the processing device, a predetermined number of combined activity features by combining a portion of the time-aligned individual activity features with a portion of the time-aligned object activity features, wherein the “portion” is a subset selected according to a stored selection rule (e.g., top-K by feature importance, threshold-based inclusion, or a preconfigured feature mask). Further, in some embodiments, combining includes concatenating features into a fixed-dimension vector and applying normalization using stored scaling parameters.

Further, in some embodiments, the method may include processing, using the processing device, a fixed-dimension feature vector corresponding to the predetermined number of combined activity features using a sequence machine learning model. Further, in some embodiments, the sequence machine learning model comprises an attention-based model configured to ingest a sequence of fixed-dimension vectors over a temporal window and produce outputs for event detection and timing. Further, the method may include producing a plurality of outputs from the sequence machine learning model. Further, in some embodiments, the plurality of outputs includes at least one of (i) an event classification label, (ii) a predicted event time offset relative to a current time stamp, (iii) a predicted performance score, and (iv) a confidence score. Further, the method may include determining, using the processing device and based on the plurality of outputs, a presentation time and a content parameter for the feedback data. Further, in some embodiments, the presentation time is computed as a sum of a current time stamp and the predicted event time offset, and the processing device schedules presentation to align with playback timing at the user device. Further, in some embodiments, the content parameter includes an overlay type, overlay position, overlay duration, text message identifier, audio cue identifier, or haptic pattern identifier. Further, the method may include generating the feedback data to include an instruction that causes the user device to present feedback at the presentation time using the content parameter. Further, in some embodiments, the instruction includes at least: (i) a rendering time stamp, (ii) a payload identifying the overlay type, and (iii) parameters defining placement and duration, enabling deterministic presentation at the user device and improving real-time usability of feedback.

Further, in some embodiments, the method may include determining, using the processing device, at least one real-time transmission parameter for the real-time communication protocol based on one or more outputs of the AI module. Further, in some embodiments, the at least one real-time transmission parameter includes an update rate, packetization interval, encoding level, redundancy level, or retransmission policy, each represented as a stored enumerated value or numeric parameter. Further, the method may include controlling, using the communication device, transmission of the feedback data according to the at least one real-time transmission parameter to satisfy a predetermined latency threshold. Further, in some embodiments, controlling includes selecting between (i) a high-priority mode used when the AI module indicates a high-confidence or imminent event and (ii) a low-priority mode used when confidence is lower or transport is congested, wherein the high-priority mode uses at least one of increased update rate or increased redundancy. Further, the method may include transmitting, using the communication device, the feedback data to the user device according to the at least one real-time transmission parameter. Further, in some embodiments, the technical effect includes improved delivery of time-sensitive feedback under variable network conditions by adapting transport parameters using AI outputs rather than transmitting a fixed-rate stream.

Further, in some embodiments, the method may include obtaining, using the processing device, at least one prior activity signature associated with the user, wherein the prior activity signature includes at least one prior reduced feature representation and at least one stored baseline statistic derived from the prior reduced feature representation. Further, in some embodiments, the prior activity signature is stored as a template vector, a distribution of feature values, or a set of time-series descriptors. Further, the method may include comparing, using the processing device, a reduced feature representation generated from the activity data with the at least one prior activity signature to determine a deviation value. Further, in some embodiments, the deviation value is computed using a distance measure (e.g., weighted L1/L2 distance), similarity score (e.g., cosine similarity), correlation score, or anomaly score generated by an anomaly detector. Further, the method may include generating, using the processing device, the feedback data based on the deviation value. Further, the deviation value may be used to select at least one of a sensitivity of the AI module or a granularity of the reduced feature representation to maintain operation within a predetermined latency threshold. Further, in some embodiments, sensitivity includes a confidence threshold or decision threshold used to trigger feedback, and granularity includes the number of features included per update and/or quantization precision for transmitted features. Further, in some embodiments, when deviation exceeds a stored deviation threshold, the processing device increases granularity for a limited duration to refine feedback, and when deviation is below the threshold, the processing device reduces granularity to conserve bandwidth and maintain latency.

Further, in some embodiments, the method may include measuring, using the communication device, one or more real-time transport metrics associated with the real-time communication protocol, including at least one of jitter, packet loss, one-way delay, round-trip time, throughput estimate, or buffer occupancy. Further, in some embodiments, the communication device computes transport metrics over a rolling measurement interval and stores the computed metrics in memory for use by the processing device. Further, the method may include adapting, using the processing device, generation of the feedback data based on the one or more real-time transport metrics. Further, adapting may include selecting a reduced feature subset according to a stored feature-priority list and/or selecting a reduced update rate by increasing an update interval. Further, in some embodiments, when packet loss or jitter exceeds a stored threshold, the processing device (i) reduces the number of transmitted features and (ii) reduces update rate to stabilize transport, and when metrics return below threshold, the processing device restores a larger feature subset and/or higher update rate. Further, the method may include transmitting, using the communication device, the feedback data using the selected reduced feature subset or the selected reduced update rate. Further, in some embodiments, the technical effect includes improved robustness and timeliness of feedback delivery under fluctuating network conditions by adapting payload and rate based on measured transport metrics.

Further, in some embodiments, the method may include executing, using the processing device, at least a portion of the AI module at an edge-computing device in communication with the communication device, wherein the edge-computing device is geographically or topologically closer to the data source device than a remote cloud system. Further, in some embodiments, executing at the edge includes running object detection and feature extraction at the edge to reduce upstream bandwidth and reduce inference delay. Further, the method may include generating, using the edge-computing device, the feedback data and a reduced feature representation derived from the activity data. Further, the method may include transmitting, using the communication device, the feedback data and the reduced feature representation to the user device. Further, the method may include withholding transmission of at least a portion of raw activity data to reduce bandwidth usage and satisfy a latency threshold associated with the real-time communication protocol. Further, in some embodiments, withholding is performed according to a stored privacy policy that specifies which raw modalities are not transmitted (e.g., raw video frames) while permitting derived features (e.g., kinematics and event labels). Further, in some embodiments, the technical effect includes reduced network utilization and reduced end-to-end latency by performing feature extraction locally and transmitting feature-level data instead of raw data.

Further, in some embodiments, the machine learning models described herein are trained using labeled training data and stored model weights in memory. Further, in some embodiments, training uses supervised learning with an objective function configured to minimize classification error for event labels and minimize timing prediction error for event time offsets. Further, during inference, the processing device applies the stored model weights to generate outputs and applies post-processing including thresholding, smoothing, or temporal aggregation using stored parameters.

Further, in some embodiments, an “activity” refers to a time-extended action or sequence of actions performed by at least one entity, such as a user or an object, and may include physical activity, sporting activity, training activity, rehabilitation activity, or any other measurable activity. Further, “performance of the activity” refers to an instance of the activity occurring over a time interval and represented in activity data as time-stamped samples. Further, in some embodiments, “activity data” refers to time-stamped measurements captured during performance of an activity. Further, the activity data may include one or more sensor modalities, including image frames, depth frames, infrared frames, inertial measurements, motion capture measurements, location samples, audio samples, pressure measurements, or combinations thereof. Further, the activity data may include metadata such as a sampling rate, a frame rate, time stamps, sensor identifiers, or calibration data. Further, in some embodiments, a “real-time communication protocol” refers to a communication protocol configured to deliver time-sensitive messages under latency constraints, such as by supporting periodic updates, low-latency delivery, and/or transport metric reporting. Further, the real-time communication protocol may support a session establishment procedure, packetization rules, message prioritization, and delivery acknowledgments. Further, in some embodiments, the protocol includes one or more transport mechanisms configured for low-latency communication.

Further, in some embodiments, an “AI module” refers to one or more software routines and/or hardware circuits that implement an inference pipeline to transform input activity data (or derived features) into outputs used for generating feedback data. Further, in some embodiments, a “machine learning model” refers to a parameterized function having stored model parameters (e.g., weights) that are applied during inference to generate outputs such as labels, scores, predictions, or embeddings. Further, in some embodiments, the AI module includes (i) preprocessing operations, (ii) one or more model inference operations, and (iii) post-processing operations such as thresholding, temporal aggregation, smoothing, or scheduling.

Further, in some embodiments, any “module,” “engine,” or “component” described herein is implemented using one or more of: (i) processor-executable instructions stored in memory and executed by one or more processors of the processing device, (ii) programmable logic, or (iii) dedicated circuitry. Further, in some embodiments, the corresponding structure for any such module includes at least the processing device, memory storing instructions, and the algorithmic steps described in the Detailed Description for performing the recited function.

Further, in some embodiments, an “object” refers to a detectable entity represented in activity data. Further, an “individual” refers to an object corresponding to a person, and a “sporting object” refers to an object associated with a sporting or training context, such as a ball, racket, bat, puck, club, or other sports equipment. Further, object types may be determined by classification labels produced by an object detection and classification model.

Further, in some embodiments, “identifying” objects includes generating at least one object indicator for each detected object, wherein an object indicator includes at least one of a class label, a confidence score, and a location descriptor. Further, “classifying” includes assigning at least one class label to an object indicator using a machine learning model output or a rule-based mapping. Further, “isolating” includes generating separate data streams, feature sets, or metadata associations corresponding to the individual and the sporting object, such as generating a first time-series feature stream for the individual and a second time-series feature stream for the sporting object.

Further, in some embodiments, a “location descriptor” refers to a representation of a location of an object in the activity data, such as a bounding shape parameterization, a region of interest descriptor, a centroid coordinate, a pose keypoint set, or other spatial descriptor.

Further, in some embodiments, a “trajectory” refers to a time-series representation of at least one spatial parameter of an entity across successive time instants, such as positions over time and optionally velocities or accelerations derived from the positions. Further, “motion information” refers to one or more quantities derived from time-series spatial parameters, such as velocity, acceleration, orientation change, curvature, or relative motion between two entities.

Further, in some embodiments, a “reduced feature representation” refers to data derived from activity data that (i) includes a predetermined set of feature fields and (ii) excludes at least a portion of raw sensor samples. Further, the reduced feature representation may be encoded as a bounded-length message format, a fixed-dimension vector, a quantized vector, or a set of feature tuples. Further, in some embodiments, the reduced feature representation includes at least one of kinematic features, temporal features, interaction features, event features, confidence values, and time stamps.

Further, in some embodiments, a “feature” refers to a numeric or categorical quantity derived from activity data, such as a position value, velocity value, pose angle, event label, confidence score, or timing offset. Further, a “feature value” refers to a value of the feature at a time instant or over a time window. Further, a “feature vector” refers to a collection of feature values arranged in a defined order and dimension.

Further, in some embodiments, “predetermined” refers to a value selected prior to execution of a step and stored in memory, configured during setup, negotiated during session establishment, or selected from a stored profile. Further, a “defined number” refers to a number that is stored in memory or determinable from stored configuration, such that the number is objectively ascertainable at runtime. Further, in some embodiments, a “predetermined number of features” refers to a fixed count specified by a configuration parameter or specified by a stored feature mask or top-K selection rule.

Further, in some embodiments, a “portion” refers to a subset selected from a larger set according to a stored and reproducible rule. Further, the stored rule may include (i) selecting the top-K features based on feature importance weights, (ii) selecting features whose confidence values exceed a threshold, (iii) selecting features identified by a predefined feature mask, or (iv) selecting features prioritized by a stored priority list.

Further, in some embodiments, a “temporal window” refers to a bounded time interval defined by a start time and end time, or by a duration parameter stored in memory. Further, a “sliding window” refers to a temporal window that advances over time by a step size. Further, “time-aligning” refers to mapping multiple feature streams to a common time base, and may include resampling, interpolation, or hold-last-sample operations, using time stamps associated with each feature stream.

Further, in some embodiments, “correlating” features refers to computing a relationship between two feature streams or feature sets based on temporal alignment. Further, correlating may include computing cross-correlation, computing similarity scores, pairing feature values by matching time stamps, or computing interaction features such as distances, relative velocities, or timing offsets.

Further, in some embodiments, “combining” features refers to producing a composite representation using two or more features or feature sets. Further, combining may include concatenation, weighted summation, normalization followed by concatenation, dimensionality reduction, or application of a feature transformation to produce a fixed-dimension vector.

Further, in some embodiments, an “output” of a machine learning model refers to one or more values produced by the model during inference. Further, outputs may include a classification label, a confidence value, a probability distribution, a regression value, an embedding vector, a timing parameter, or a performance metric.

Further, in some embodiments, “feedback data” refers to data transmitted to cause a user device to present feedback corresponding to the activity. Further, feedback data may include at least one of a feedback message, a score, a predicted metric, a corrective instruction, an overlay instruction, an audio cue identifier, a haptic cue identifier, or a presentation schedule. Further, in some embodiments, feedback data includes parameters enabling deterministic presentation, such as a time stamp, a time offset, a display duration, and a placement descriptor.

Further, in some embodiments, a “presentation time” refers to a time value or offset indicating when feedback is to be presented by a user device. Further, “synchronizing” refers to coordinating the presentation time with the activity such that feedback is presented contemporaneously with the relevant portion of the activity, using time stamps, offsets, or a shared clock reference.

Further, in some embodiments, a “latency threshold” or “latency budget” refers to a stored or negotiated maximum allowable end-to-end delay for feedback delivery. Further, “end-to-end latency” refers to a measured delay between (i) receipt of activity data at the communication device or processing device and (ii) receipt and/or presentation of feedback at the user device. Further, in some embodiments, end-to-end latency is computed based on time stamps inserted into messages and measured at defined points of the pipeline.

Further, in some embodiments, “transport metrics” refer to measured or estimated network and protocol performance parameters, including at least one of jitter, packet loss, one-way delay, round-trip time, throughput estimate, retransmission count, buffer occupancy, or congestion indicator.

Further, in some embodiments, a “transmission parameter” refers to a parameter controlling how data is transmitted using the real-time communication protocol. Further, transmission parameters may include update rate, packetization interval, payload size target, prioritization level, redundancy level, encoding level, retransmission policy, forward-error-correction selection, or scheduling policy.

Further, in some embodiments, “controlling operation of the communication device” includes selecting or adjusting one or more transmission parameters and applying the selected parameters to message scheduling, packetization, encoding, redundancy, or prioritization. Further, controlling may be performed dynamically based on transport metrics, AI outputs, or both, to satisfy latency and/or bandwidth goals.

Further, in some embodiments, an “edge-computing device” refers to a computing device positioned closer to a data source device and/or user device than a remote cloud system, such that processing at the edge reduces transport delay. Further, an edge-computing device may include a gateway device, router, access point, base station, local server, or other intermediate compute node.

Further, in some embodiments, a “privacy policy” refers to a stored rule set indicating which portions of activity data are permitted to be transmitted, which portions are restricted, and what derived features are permitted. Further, “withholding transmission” refers to not transmitting restricted raw data, redacting restricted raw data, or replacing restricted raw data with derived features that satisfy the privacy policy.

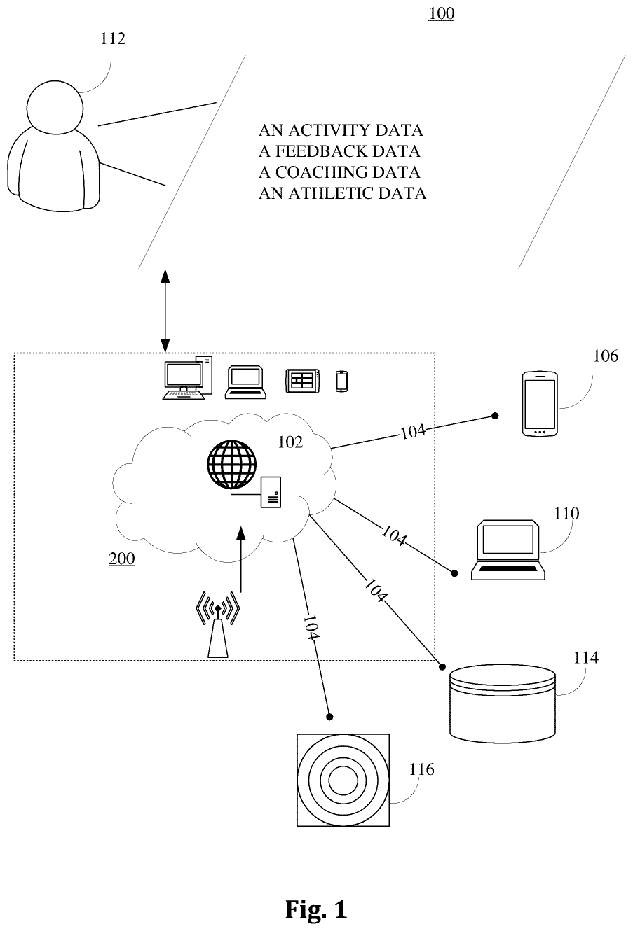

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 200.

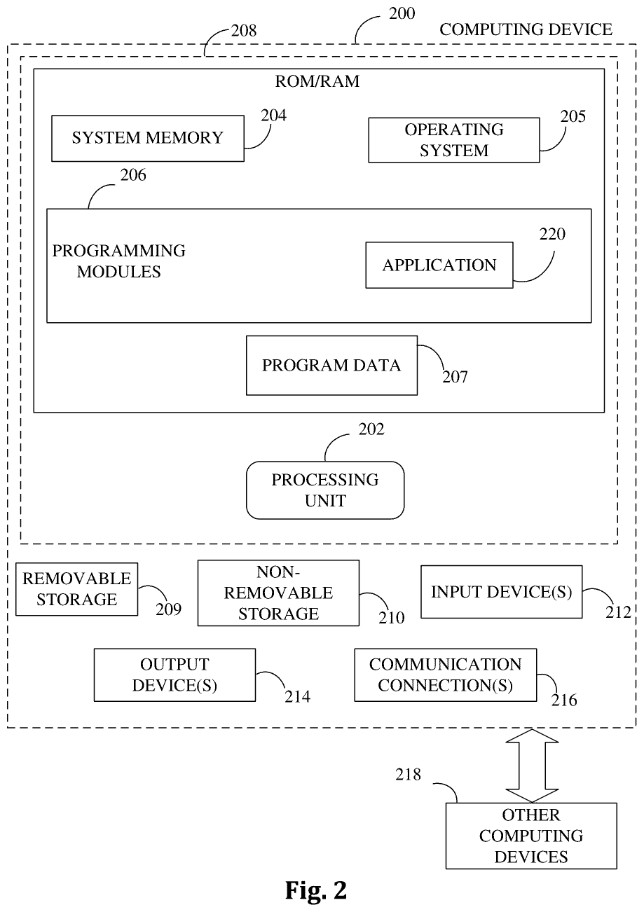

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g. random-access memory (RAM)), non-volatile (e.g. read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods'stages may be modified in any manner, including by reordering stages and/or inserting or deleting stages, without departing from the disclosure.

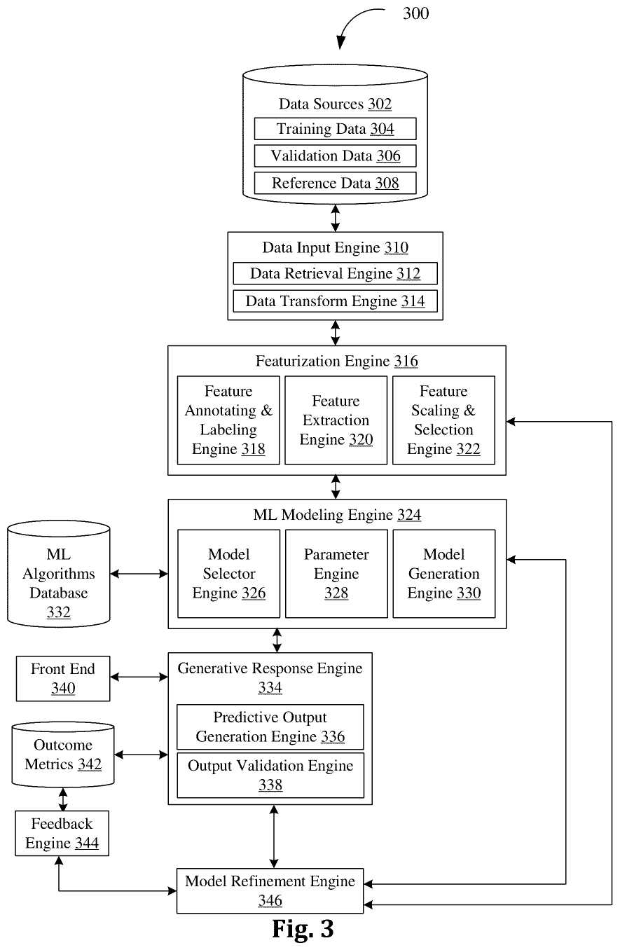

FIG. 3 is a block diagram illustrating a machine-learning system 300 for implementing various embodiments of this disclosure, in accordance with some embodiments. Although the disclosed machine-learning system 300 depicts particular system components and an arrangement of such components, the given depiction is to facilitate a discussion of the present technology and should not be considered limiting unless specified in the appended claims. For example, some components that are illustrated as separate may be combined with other components, and some components may be divided into separate components.

Accordingly, the machine-learning system 300 may include a plurality of interrelated modules and engines configured to implement a machine-learning pipeline. Further, the machine-learning system 300 may include a data sources module 302 that is made up of a training data repository 304, a validation data repository 306, and a reference data repository 308, each repository being configured to store respective classes of input records and reference information. Further, the machine-learning system 300 may include a data input engine 310 configured to receive data from the data sources module 302. Further, the data input engine 310 may include a data retrieval engine 312 configured to access and ingest data from the repositories (304, 306, 308), and a data transform engine 314 configured to perform initial normalization, parsing, and format conversion on the ingested data. Further, the machine-learning system 300 may include a featurization engine 316 configured to prepare temporal and predictive representations of transformed data. Further, the featurization engine 316 may include a feature annotating & labeling engine 318 for applying labels and annotations to data instances, a feature extraction engine 320 for deriving feature vectors and candidate predictors, and a feature scaling & selection engine 322 for performing numerical scaling, dimensionality reduction, and selection of salient features. Further, the machine-learning system 300 may include a machine learning (ML) modeling engine 324 configured to construct predictive models from selected features. Further, the ML modeling engine 324 may include a model selector engine 326 for selecting among candidate model classes, a parameter engine 328 for determining and tuning hyper parameters, and a model generation engine 330 for instantiating and training model artifacts according to selected architectures and parameters. Further, the machine-learning system 300 may include an ML algorithms database 332 configured to store algorithmic implementations, model templates, and associated metadata and to be accessible by components of the ML modeling engine 324. Further, the machine-learning system 300 may include a generative response engine 334 configured to produce user-facing outputs based on the trained models. Further, the generative response engine 334 may include a predictive output generation engine 336 for generating predictions or synthesized responses and an output validation engine 338 for verifying, filtering, and validating generated outputs against predefined criteria and reference data. Further, the machine-learning system 300 may include a front end 340 configured to present validated outputs to end users and to collect interaction signals. Further, the machine-learning system 300 may include an outcome metrics module 342 configured to compute performance measures, accuracy statistics, and other evaluation metrics derived from model outputs and user interactions. Further, the machine-learning system 300 may include a feedback engine 344 configured to aggregate outcome metrics and user feedback and to format such information for reuse. Further, the machine-learning system 300 may include a model refinement engine 346 configured to receive feedback from the feedback engine 344 and the outcome metrics module 342, and to effect iterative updates to the ML modeling engine 324 and to the ML algorithms database 332. Further, the components are communicatively coupled so that data and control signals are exchanged among the repositories (304, 306, 308), the data input engine 310, the featurization engine 316, the ML modeling engine 324 (with algorithmic support from the ML algorithms database 332), the generative response engine 334 and the front end 340 for output generation. Further, the outcome metrics 342 and feedback engine 344 provide closed-loop signals to the model refinement engine 346 to enable retraining, parameter adjustment, and algorithm selection, thereby enabling cooperative execution of data acquisition, feature engineering, model construction, output generation, validation, evaluation, and iterative refinement within the disclosed machine-learning system 300.

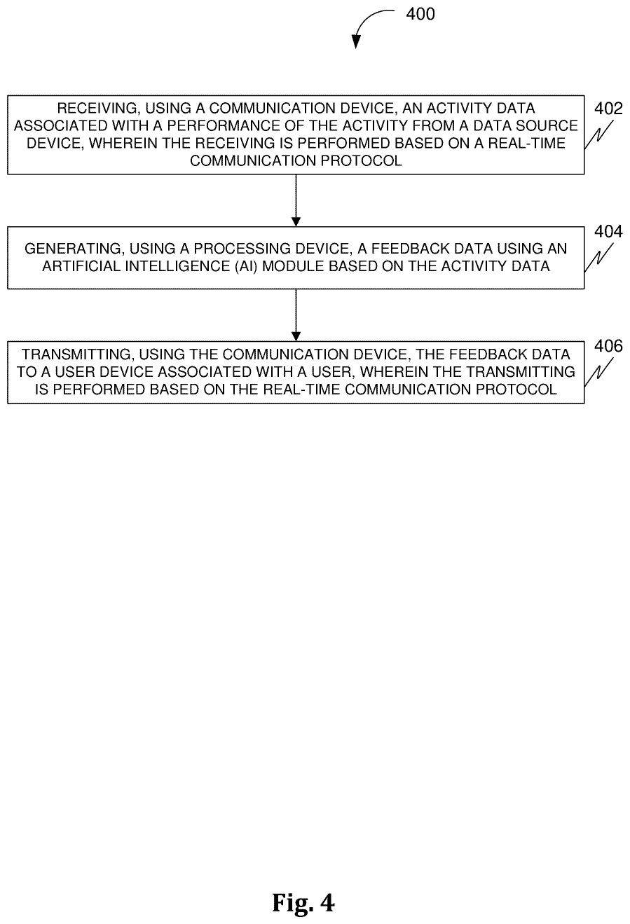

FIG. 4 illustrates a flowchart of a method 400 of provisioning a feedback based on an activity, in accordance with some embodiments. Accordingly, the method 400 may include a step 402 of receiving, using a communication device 502, an activity data associated with a performance of the activity from a data source device 506. Further, the receiving may be performed based on a real-time communication protocol. Further, the method 400 may include a step 404 of generating, using a processing device 504, a feedback data using an artificial intelligence (AI) module based on the activity data. Further, the method 400 may include a step 406 of transmitting, using the communication device 502, the feedback data to a user device 508 associated with a user. Further, the transmitting may be performed based on the real-time communication protocol.

In some embodiments, the activity data includes a video data representing a visual content associated with the activity.

In some embodiments, the activity may be associated with two or more objects. Further, the feedback data includes an object data representing an object associated with the activity. Further, the object may be a sport equipment.

In some embodiments, the user includes one or more of an athlete 802 and a coach 804. Further, the user device 508 includes an athlete device associated with the athlete 802 and a coach device associated with the coach 804. Further, the performance of the activity includes performance of the activity by the athlete 802. Further, the transmitting of the feedback data to the user device 508 includes transmitting the feedback data to the coach device in a first time period. Further, the method 400 further includes receiving, using the communication device 502, a coaching data from the coach device, Further, the coaching data represents a coaching session. Further, the receiving may be based on the real-time communication protocol. Further, the generating of the feedback data may be further based on the coaching data. Further, the transmitting of the feedback data to the user device 508 further includes transmitting the feedback data to the athlete device in a second time period. Further, the second time period occurs later than the first time period.

In some embodiments, the athlete device includes one or more of a first athlete device associated with a first athlete and a second athlete device associated with a second athlete. Further, the method 400 further includes receiving, using the communication device 502, an athletic data from the first athlete device. Further, the receiving may be based on the real-time communication protocol. Further, the generating of the feedback data may be further based on the athletic data. Further, the transmitting of the feedback data to the user device 508 includes transmitting of the feedback data to the second athlete device. Further, the transmitting may be based on the real-time communication protocol.

In some embodiments, the feedback data includes an insight data corresponding to an insight on the performance of the activity. Further, the feedback data further includes the video data embedded with the insight data.

In some embodiments, the video data includes two or more video frames. Further, each of the two or more video frames may be associated with two or more time instances associated with the activity. Further, the insight data includes two or more insight data corresponding to the two or more time instances.

In some embodiments, the activity data includes two or more activity data corresponding to two or more activity phases associated with the activity. Further, the two or more activity data includes a first activity data representing a first activity phase associated with a first time period and a second activity data representing a second activity phase associated with a second time period. Further, the second time period occurs later than the first time period. Further, the method 400 further includes determining, using the processing device 504, a transition data representing one or more transitions between the first activity phase and the second activity phase. Further, the generating of feedback data may be further based on the transition data. Further, the two or more activity phases include a shot phase associated with the activity. Further, the shot phase includes one or more of a transfer phase, a pocket phase, and a release phase.

In some embodiments, the activity data further includes two or more phase metrics data corresponding to each of the two or more activity phases. Further, the two or more phase metrics data includes one or more of a reference phase metric data and a current phase metric data. Further, the reference phase metric data represents a standard metric value for the performance of the activity. Further, the current phase metric data represents a metric value generated based on the performance of the activity. Further, the method 400 further includes analyzing, using the processing device 504, each of the reference phase metric data and the current phase metric data. Further, the generating of the feedback data may be further based on the analyzing of the reference phase metric data and the current phase metric data.

In some embodiments, the activity data includes an activity factor data representing a factor affecting the performance by the user. Further, the activity factor data includes a first activity factor data associated with a first time period and a second activity factor data associated with a second time period. Further, the second time period occurs later than the first time period. Further, the method 400 further includes generating, using the processing device 504, an adaptive score data based on each of the first activity factor data and the second activity factor data. Further, the adaptive score data represents a score associated with the performance of the activity. Further, the feedback data includes the adaptive score data.

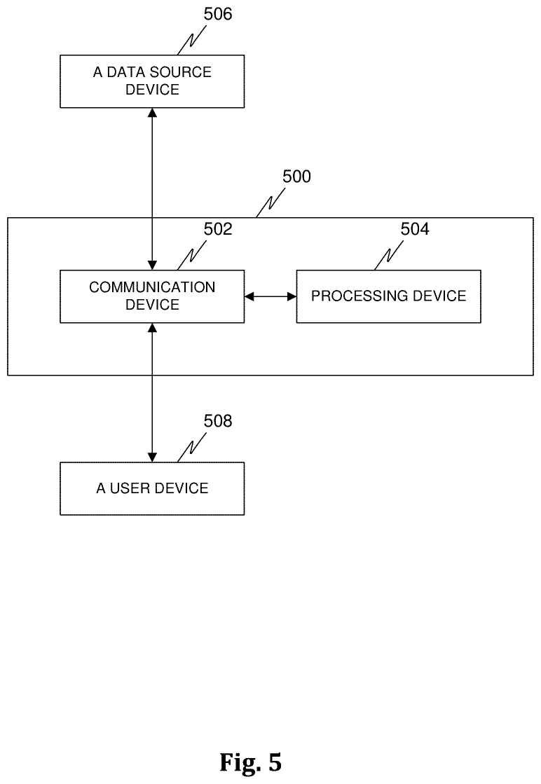

FIG. 5 illustrates a block diagram of a system 500 for provisioning a feedback based on an activity, in accordance with some embodiments. Accordingly, the system 500 may include a communication device 502. Further, the communication device 502 may be configured for receiving an activity data associated with a performance of the activity from a data source device 506. Further, the receiving may be performed based on a real-time communication protocol. Further, the communication device 502 may be configured for transmitting a feedback data to a user device 508 associated with a user. Further, the transmitting may be performed based on the real-time communication protocol. Further, the system 500 may include a processing device 504 communicatively coupled with the communication device 502. Further, the processing device 504 may be configured for generating the feedback data using an artificial intelligence (AI) module based on the activity data.

In some embodiments, the activity data includes a video data representing a visual content associated with the activity.

In some embodiments, the activity may be associated with two or more objects. Further, the feedback data includes an object data representing an object associated with the activity. Further, the object may be a sport equipment.

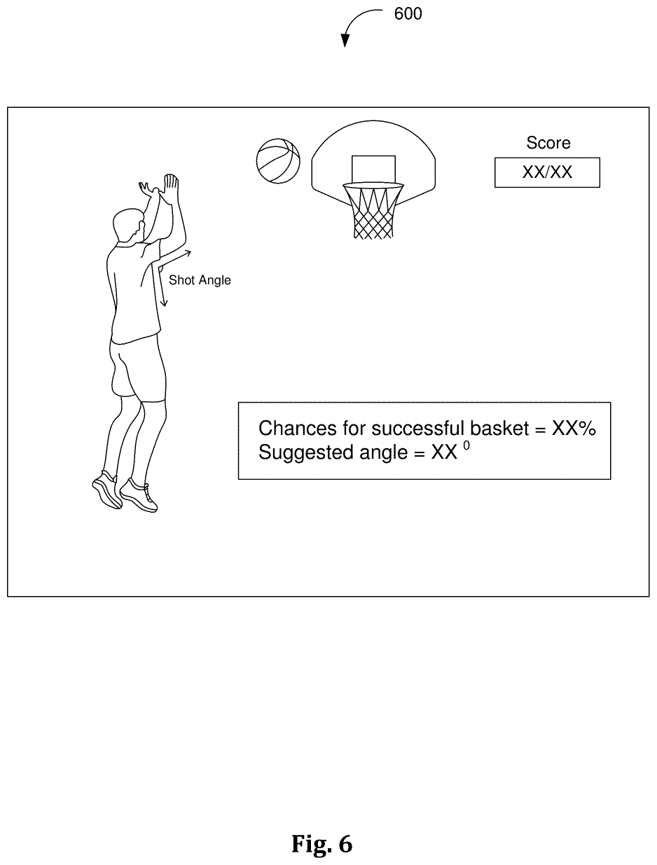

FIG. 6 is an illustration of a user interface 600 of a user presentation device showing an integration of one or more of scorecards, video overlays, and real-time communication technologies for feedback delivery, in accordance with some embodiments. Further, in some embodiments, the user includes one or more of an athlete 802 and a coach 804. Further, the user device 508 includes an athlete device associated with the athlete 802 and a coach device associated with the coach 804. Further, the performance of the activity includes performance of the activity by the athlete 802. Further, the transmitting of the feedback data to the user device 508 includes transmitting the feedback data to the coach device in a first time period. Further, the communication device 502 may be further configured for receiving a coaching data from the coach device. Further, the coaching data represents a coaching session. Further, the receiving may be based on the real-time communication protocol. Further, the generating of the feedback data may be further based on the coaching data. Further, the transmitting of the feedback data to the user device 508 further includes transmitting the feedback data to the athlete device in a second time period. Further, the second time period occurs later than the first time period.

In some embodiments, the athlete device includes one or more of a first athlete device associated with a first athlete and a second athlete device associated with a second athlete. Further, the communication device 502 may be further configured for receiving an athletic data from the first athlete device. Further, the receiving may be based on the real-time communication protocol. Further, the generating of the feedback data may be further based on the athletic data. Further, the transmitting of the feedback data to the user device 508 includes transmitting of the feedback data to the second athlete device. Further, the transmitting may be based on the real-time communication protocol.

In some embodiments, the feedback data includes an insight data corresponding to an insight on the performance of the activity. Further, the feedback data further includes the video data embedded with the insight data.

In some embodiments, the video data includes two or more video frames. Further, each of the two or more video frames may be associated with two or more time instances associated with the activity. Further, the insight data includes two or more insight data corresponding to the two or more time instances.

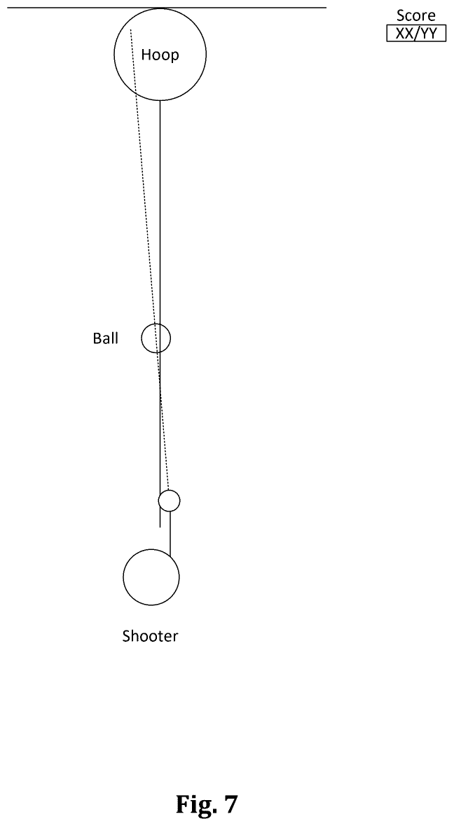

FIG. 7 illustrates a three-dimensional top down view during a release phase in a sport of basketball, in accordance with some embodiments. Further, in some embodiments, the activity data includes two or more activity data corresponding to two or more activity phases associated with the activity. Further, the two or more activity data includes a first activity data representing a first activity phase associated with a first time period and a second activity data representing a second activity phase associated with a second time period. Further, the second time period occurs later than the first time period. Further, the processing device 504 may be further configured for determining a transition data representing one or more transitions between the first activity phase and the second activity phase. Further, the generating of the feedback data may be further based on the transition data. Further, the two or more activity phases include a shot phase associated with the activity. Further, the shot phase includes one or more of a transfer phase, a pocket phase, and a release phase.

In some embodiments, the activity data further includes two or more phase metrics data corresponding to each of the two or more activity phases. Further, the two or more phase metrics data includes one or more of a reference phase metric data and a current phase metric data. Further, the reference phase metric data represents a standard metric value for the performance of the activity. Further, the current phase metric data represents a metric value generated based on the performance of the activity. Further, the processing device 504 may be further configured for analyzing each of the reference phase metric data and the current phase metric data. Further, the generating of the feedback data may be further based on the analyzing of the reference phase metric data and the current phase metric data.

In some embodiments, the activity data includes an activity factor data representing a factor affecting the performance by the user. Further, the activity factor data includes a first activity factor data associated with a first time period and a second activity factor data associated with a second time period. Further, the second time period occurs later than the first time period. Further, the processing device 504 may be further configured for generating an adaptive score data based on each of the first activity factor data and the second activity factor data. Further, the adaptive score data represents a score associated with the performance of the activity. Further, the feedback data includes the adaptive score data.

In some embodiments, the activity data further includes an audio data representing a vocal content associated with the activity.

In some embodiments, the activity data includes a sensor data associated with a sensor. Further, the sensor data represents a variable attribute associated with the activity.

In some embodiments, the sensor includes a wearable sensor which may be configured to be worn by the user. Further, the variable attribute includes a user-based attribute associated with the user relative to the activity.

In some embodiments, the wearable sensor includes an accelerometer. Further, the variable attribute further includes an acceleration associated with the user relative to the activity.

In some embodiments, the variable attribute further includes a movement pattern representing a movement of the user relative to the activity.

In some embodiments, the wearable sensor further includes a gyroscope. Further, the variable attribute further includes an orientation associated with the user relative to the activity.

In some embodiments, the wearable sensor further includes a heart rate sensor. Further, the variable attribute further includes a heart rate of the user relative to the activity.

In some embodiments, the wearable sensor further includes an EMG sensor. Further, the variable attribute further includes an electrical activity of the muscle of the user relative to the activity.

In some embodiments, the sensor further includes an external sensor placed in an environment associated with the user.

In some embodiments, the external sensor includes a tracking sensor which may be configured for detecting one or more of a speed, a distance, and a position of the user relative to the activity.

In some embodiments, the external sensor further includes a pressure sensor. Further, the variable attribute includes a pressure exerted by the user relative to the performance of the activity.

In some embodiments, the activity data further includes a meta data corresponding to a technical information associated with the data source device 506.

In some embodiments, the technical information includes one or more of a source of the activity data, a date associated with the activity data, and a time of collection of the activity data.

In some embodiments, the transmitting of the feedback data may be further based on two or more feedback delivery channels.

In some embodiments, the activity includes a sporting event.

In some embodiments, the sporting event includes one or more of a swimming session, a gymnastic session, cycling, weightlifting, tennis, basketball, football, cricket, baseball, and an athletic event.

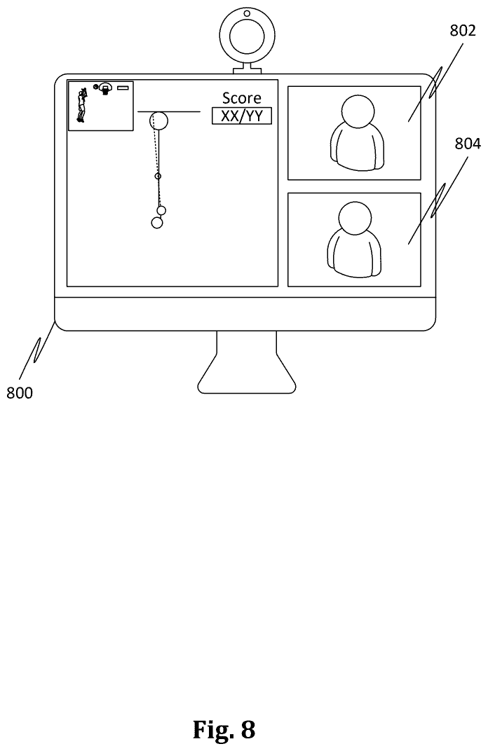

FIG. 8 illustrates a user presentation device 800 showing a remote, collaborative coaching platform, in accordance with some embodiments. Further, in some embodiments, the activity further includes one or more of a yoga session, a martial art event, a dance session, a music session, an art session, a cooking session, and a public speaking session.

In some embodiments, the activity data further includes an athlete mechanics data representing a mechanical action associated with the athlete 802.

In some embodiments, the real-time communication protocol includes one or more of a WebRTC protocol, a WebSocket Protocol, a real-time transport protocol, a real-time messaging protocol, a VoI protocol, an XMP protocol, a MQTT protocol, and an HLS protocol.

In some embodiments, the feedback data further includes a scorecard data representing a score associated with the activity.

In some embodiments, the feedback data includes the video data overlaid with the insight data.

In some embodiments, the feedback data includes an enhanced video data based on an integration of the insight data into the video data.

In some embodiments, the feedback data includes an instruction data representing an instruction for the activity.

In some embodiments, the activity factor data includes a performance data representing the performance of the user.

In some embodiments, the performance data includes a point data representing a point scored by the user.

In some embodiments, the performance data includes a speed data representing a speed of the user.

In some embodiments, the performance data an accuracy data represent an accuracy of the user.

In some embodiments, the activity factor data further includes a skill level data representing a skill level of the user.

In some embodiments, the activity factor data further includes a condition data representing a condition affecting the performance of the user.

In some embodiments, the condition data includes an environmental condition data representing an environmental condition associated with the performance of the activity.

In some embodiments, the condition data further includes a time data corresponding to a time of a day associated with the activity.

In some embodiments, the condition data includes a field condition data representing a field condition associated with the activity.

In some embodiments, the activity factor data further includes a historical data corresponding to a previous performance record of the user.

In some embodiments, the historical data includes a past performance record data representing one or more of a past achievement and a past score associated with the user.

In some embodiments, the historical data further includes a statistical data representing a statistic of one or more of a current achievement and a past achievement associated with the user.

In some embodiments, the user includes one or more of a player and a team.

In some embodiments, the coach device includes each of a first coach device associated with a first coach and a second coach device associated with a second coach.

In some embodiments, the receiving of the coaching data from the coach device includes receiving the coaching data from the first coach device. Further, the transmitting of the feedback data to the user device 508 further includes transmitting the feedback data to the second coach device. Further, the transmitting may be based on the real-time communication protocol.

In some embodiments, the athlete device may be associated with an athlete location and the coach device may be associated with a coach location. Further, each of the athlete location and the coach location may be geographically distinct.

In some embodiments, the video data includes one or more of a live video data and a recorded video data.

In some embodiments, the insight data includes an annotation data representing one or more of an improvement and an instruction for the user based on the performance.

In some embodiments, the score further corresponds to a performance metric associated with the user based on the performance of the activity.

In some embodiments, the feedback data further includes a personalized training data representing a personalized training for the user.

In some embodiments, the sports equipment includes one or more of a ball, a hockey stick, and a baseball bat.

In some embodiments, the object data further includes a sports-mechanics data representing a mechanical characteristic of the sports equipment associated with the activity.

In some embodiments, the object data includes an object placement data corresponding to a placement of the object during the performance of the activity.

In some embodiments, the object placement data includes an object angle data representing the angle of the object during the performance of the activity.

In some embodiments, the object includes a hockey stick. Further, the angle includes the hockey stick angle during a slap shot associated with the activity.

In some embodiments, the object placement data further includes an object interaction data representing an interaction of the object with each of the two or more objects.

In some embodiments, the object data further includes one or more of an alignment data corresponding to an alignment of the object corresponding to the performance of the activity and a positional data corresponding to a position of the object.

In some embodiments, the object data further includes a grip adjustment data representing a grip adjustment for the object based on the activity.

In some embodiments, the object data includes an object placement data corresponding to a placement of the object during the performance of the activity.

In some embodiments, the object placement data includes an object angle data representing the angle of the object during the performance of the activity.

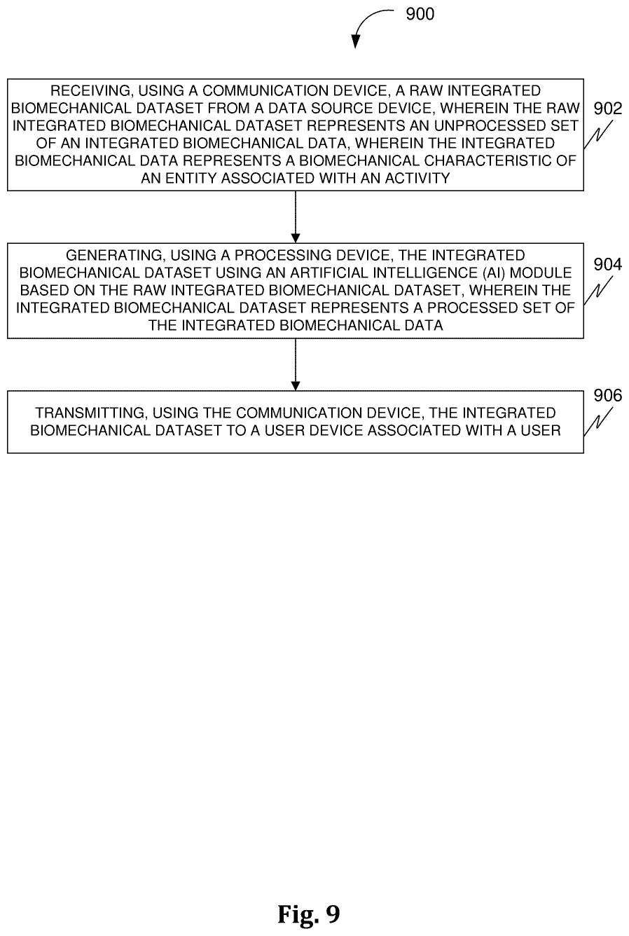

FIG. 9 illustrates a flowchart of a method 900 of provisioning an integrated biomechanical dataset, in accordance with some embodiments. Accordingly, the method 900 may include a step 902 of receiving, using a communication device 1102, a raw integrated biomechanical dataset from a data source device 1108. Further, the raw integrated biomechanical dataset represents an unprocessed set of an integrated biomechanical data. Further, the integrated biomechanical data represents a biomechanical characteristic of an entity associated with an activity. Further, the method 900 may include a step 904 of generating, using a processing device 1104, the integrated biomechanical dataset using an artificial intelligence (AI) module based on the raw integrated biomechanical dataset. Further, the integrated biomechanical dataset represents a processed set of the integrated biomechanical data. Further, the method 900 may include a step 906 of transmitting, using the communication device 1102, the integrated biomechanical dataset to a user device 1110 associated with a user.

In some embodiments, the method 900 may further include processing, using the processing device 1104, the raw integrated biomechanical dataset. Further, the generating of the integrated biomechanical dataset may be further based on the processing of the raw integrated biomechanical dataset.

In some embodiments, the integrated biomechanical data includes a 3D model data associated with a performance of the activity. Further, the 3D model data represents a 3D model of the entity associated with the activity. Further, the entity includes one or more of the user and an equipment associated with the activity. Further, the 3D model data includes one or more of a user model data representing a 3D model of the user and an equipment model data representing the 3D model of the equipment. Further, the 3D model data includes a skeletal model data representing a skeletal model comprising two or more line segments. Further, each of the two or more line segments includes two endpoints. Further, the skeletal model further includes two or more nodes. Further, each of the two or more nodes connect at least two of the two or more line segments. Further, each of the two or more line segments and the two or more nodes may be connected based on an anatomical structure of the user and the equipment.

In some embodiments, the integrated biomechanical dataset includes a set of a representation data corresponding to a representation of the raw biomechanical dataset. Further, the representation data includes one or more of a vector representation data corresponding to a vector representation of the integrated biomechanical data. Further, the integrated biomechanical data may be converted to a high-dimensional numerical vector and a knowledge graph data representing the integrated biomechanical as two or more entities and a relationship between each of the two or more entities. Further, the entity corresponds to a key feature of the integrated biomechanical data.

In some embodiments, the AI module includes a generative artificial intelligence (AI) module. Further, the method 900 further includes generating, using the processing device 1104, an insight data using the AI module based on the integrated biomechanical data associated with the integrated biomechanical dataset.

### In Some Embodiments, the Generative Ai Module Includes Two or More Artificial

intelligence (AI) modules.

In some embodiments, the insight data includes a spatial characteristic data representing a spatial characteristic of an object associated with the activity. Further, the spatial characteristic may be associated with a six-dimensional space corresponding to a six-dimensional representation of the object. Further, the two or more AI modules include a spatio-temporal AI module which may be configured for generating the spatial characteristic associated with the object. Further, the spatial characteristic includes one or more of a rotational characteristic and a positional characteristic. Further, the rotational characteristic corresponds to a rotation motion associated with the object during the performance of the activity. Further, the positional characteristic corresponds to a position of the object in relation to an environment in which the activity may be performed.

In some embodiments, the insight data includes two or more performance metrics data corresponding to each of the two or more activity phases. Further, the two or more performance metrics data represents a metric value generated from each of the two or more activity phases. Further, the two or more performance metrics data may be based on each of a reference metric data and a current metric data. Further, the reference metric data represents a standard metric value for the performance of the activity. Further, the current metric data represents a metric value generated based on the performance of the activity. Further, the insight data includes a personalized metric data. Further, the generating of the insight data includes generating of the personalized metric data based on each of a reference metric data and a current metric data associated with the entity.

In some embodiments, the user device 1110 includes a user presentation device, which may be configured for presenting the insight data. Further, the user device 1110 further includes a user input device, which may be configured for generating an additional information data representing an additional information associated with the performance of the activity. Further, the user device 1110 further includes a user communication device, which may be configured for transmitting the additional information data to the communication device 1102.

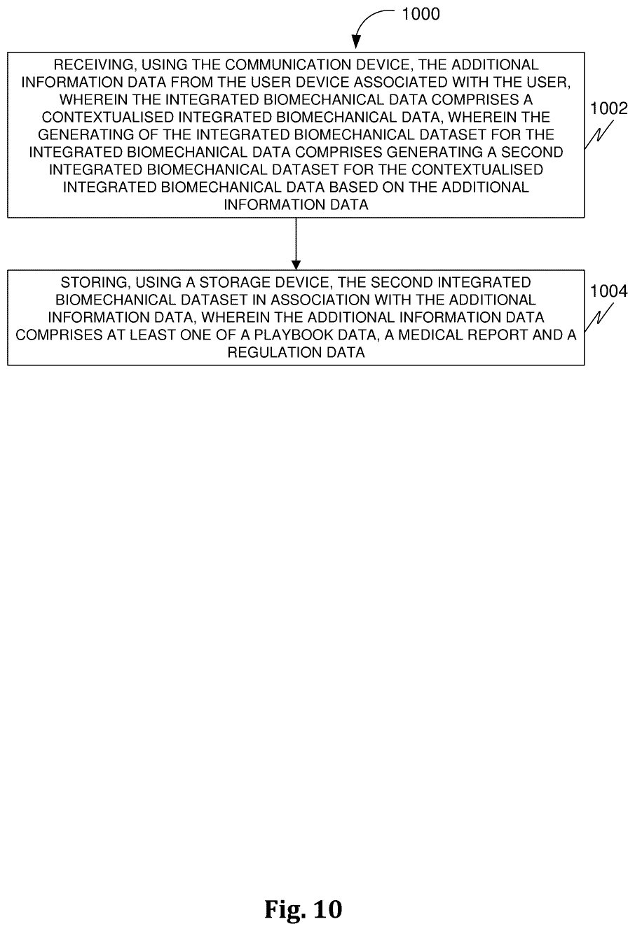

FIG. 10 illustrates a flowchart of a method 1000 of provisioning an integrated biomechanical dataset including receiving, using the communication device 1102, the additional information data from the user device 1110 associated with the user, in accordance with some embodiments. Further, in some embodiments, the method 1000 may include a step 1002 of receiving, using the communication device 1102, the additional information data from the user device 1110 associated with the user. Further, the integrated biomechanical data includes contextualized integrated biomechanical data. Further, the generating of the integrated biomechanical dataset for the integrated biomechanical data includes generating a second integrated biomechanical dataset for the contextualized integrated biomechanical data based on the additional information data. Further, in some embodiments, the method 1000 may include a step 1004 of storing, using a storage device 1106, the second integrated biomechanical dataset in association with the additional information data. Further, the additional information data includes one or more of a playbook data, a medical report and a regulation data.

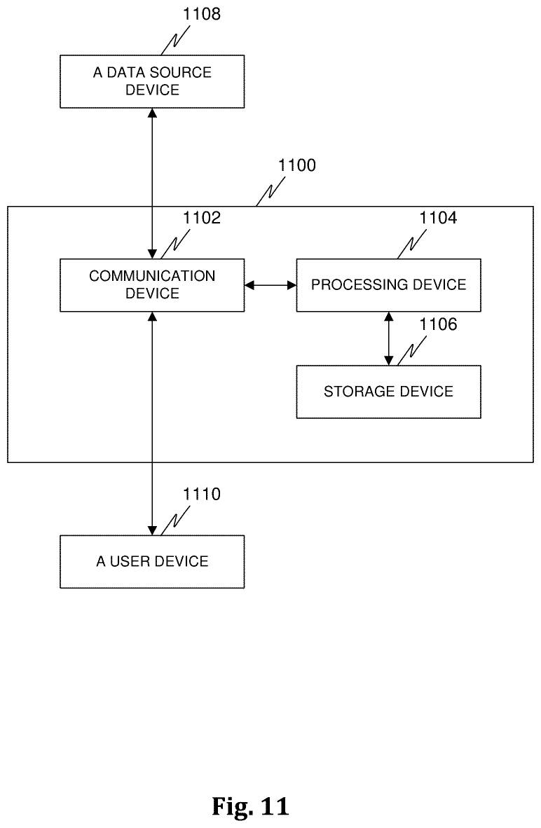

FIG. 11 illustrates a block diagram of a system 1100 of provisioning an integrated biomechanical dataset, in accordance with some embodiments. Accordingly, the system 1100 may include a communication device 1102. Further, the communication device 1102 may be configured for receiving a raw integrated biomechanical dataset from a data source device 1108. Further, the raw integrated biomechanical dataset represents an unprocessed set of an integrated biomechanical data. Further, the integrated biomechanical data represents a biomechanical characteristic of an entity associated with an activity. Further, the communication device 1102 may be configured for transmitting the integrated biomechanical dataset to a user device 1110 associated with a user. Further, the system 1100 may include a processing device 1104 communicatively coupled with the communication device 1102. Further, the processing device 1104 may be configured for generating the integrated biomechanical dataset using an artificial intelligence (AI) module based on the raw integrated biomechanical dataset. Further, the integrated biomechanical dataset represents a processed set of the integrated biomechanical data.

In some embodiments, the processing device 1104 may be further configured for processing the raw integrated biomechanical dataset. Further, the generating of the integrated biomechanical dataset may be further based on the processing of the raw integrated biomechanical dataset.

In some embodiments, the integrated biomechanical data includes a 3D model data associated with a performance of the activity. Further, the 3D model data represents a 3D model of the entity associated with the activity. Further, the entity includes one or more of the user and an equipment associated with the activity. Further, the 3D model data includes one or more of a user model data representing a 3D model of the user and an equipment model data representing the 3D model of the equipment. Further, the 3D model data includes a skeletal model data representing a skeletal model comprising two or more line segments. Further, each of the two or more line segments includes two endpoints. Further, the skeletal model further includes two or more nodes. Further, each of the two or more nodes connect at least two of the two or more line segments. Further, each of the two or more line segments and the two or more nodes may be connected based on an anatomical structure of the user and the equipment.

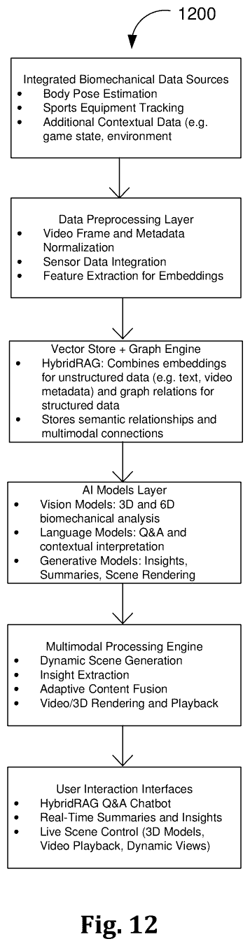

FIG. 12 illustrates an operational workflow 1200 of the system 1100 for provisioning an integrated biomechanical dataset, in accordance with some embodiments. Further, in some embodiments, the integrated biomechanical dataset includes a set of a representation data corresponding to a representation of the raw biomechanical dataset. Further, the representation data includes one or more of a vector representation data corresponding to a vector representation of the integrated biomechanical data. Further, the integrated biomechanical data may be converted to a high-dimensional numerical vector and a knowledge graph data representing the integrated biomechanical as two or more entities and a relationship between each of the two or more entities. Further, the entity corresponds to a key feature of the integrated biomechanical data.

In some embodiments, the AI module includes a generative artificial intelligence (AI) module. Further, the processing device 1104 may be further configured for generating an insight data using the AI module based on the integrated biomechanical data associated with the integrated biomechanical dataset.

In some embodiments, the generative AI module includes two or more artificial intelligence (AI) modules.

In some embodiments, the insight data includes a spatial characteristic data representing a spatial characteristic of an object associated with the activity. Further, the spatial characteristic may be associated with a six-dimensional space corresponding to a six-dimensional representation of the object. Further, the two or more AI modules include a spatio-temporal AI module which may be configured for generating the spatial characteristic associated with the object. Further, the spatial characteristic includes one or more of a rotational characteristic and a positional characteristic. Further, the rotational characteristic corresponds to a rotation motion associated with the object during the performance of the activity. Further, the positional characteristic corresponds to a position of the object in relation to an environment in which the activity may be performed.

In some embodiments, the insight data includes two or more performance metrics data corresponding to each of the two or more activity phases. Further, the two or more performance metrics data represents a metric value generated from each of the two or more activity phases. Further, the two or more performance metrics data may be based on each of a reference metric data and a current metric data. Further, the reference metric data represents a standard metric value for the performance of the activity. Further, the current metric data represents a metric value generated based on the performance of the activity. Further, the insight data includes a personalized metric data. Further, the generating of the insight data includes generating the personalized metric data based on each of a reference metric data and a current metric data associated with the entity.

In some embodiments, the user device 1110 includes a user presentation device which may be configured for presenting the insight data. Further, the user device 1110 further includes a user input device which may be configured for generating an additional information data representing an additional information associated with the performance of the activity. Further, the user device 1110 further includes a user communication device which may be configured for transmitting the additional information data to the communication device 1102.

In some embodiments, the communication device 1102 may be further configured for receiving the additional information data from the user device 1110 associated with the user. Further, the integrated biomechanical data includes a contextualized integrated biomechanical data. Further, the generating of the integrated biomechanical dataset for the integrated biomechanical data includes generating a second integrated biomechanical dataset for the contextualized integrated biomechanical data based on the additional information data. Further, the system 1100 further includes a storage device 1106 communicatively coupled with the processing device 1104. Further, the storage device 1106 may be configured for storing the second integrated biomechanical dataset in association with the additional information data. Further, the additional information data includes one or more of a playbook data, a medical report and a regulation data.

In some embodiments, the raw integrated biomechanical dataset includes a set of an activity data corresponding to a performance of an activity.

In some embodiments, the activity data includes a video data corresponding to a visual content associated with the activity.

In some embodiments, the activity includes a sporting event.

In some embodiments, the sport includes hockey.

In some embodiments, the user includes a player.

In some embodiments, the equipment includes a hockey stick.

In some embodiments, the activity includes two or more activity phases. Further, the integrated biomechanical data includes two or more integrated biomechanical data representing each of the plurality of activity phases.

In some embodiments, the integrated biomechanical data includes one or more of an activity type data representing a type of the activity performed and a user data representing the user associated with the activity.

In some embodiments, the vector representation data includes a similarity vector data corresponding to an identification of one or more of the type of the activity and the user.

In some embodiments, the insight data includes a textual data representing a textual insight relative to the performance of the activity.

In some embodiments, the insight data includes an audio data corresponding to an insight relative to the activity in an audio format.

In some embodiments, the insight data includes a 3D visualization model data corresponding to a model representing a 3D visualization of an object associated with the activity. Further, the generating of the 3D visualization data may be based on a rendering framework.

In some embodiments, the rendering framework includes one or more of a Babylon.js framework, a Three.js framework, a PixiJS framework, an A-Frame framework, a Unity framework, and an Unreal Engine.

In some embodiments, the method 900 may further include determining, using the processing device 1104, one or more of an intent and a context of the integrated biomechanical data. Further, the processing of the raw integrated biomechanical dataset may be further based on determining one or more of the intent and the context of the integrated biomechanical data. Further, the processing of the raw integrated biomechanical dataset may be further based on applying one or more of a predefined rule and a safety guideline to the integrated biomechanical data using a guardrail application.

In some embodiments, the method 900 may further include analyzing, using the processing device 1104, the insight data. Further, the analyzing may be based on a guardrail application. Further, the analyzing of the insight data corresponds to determining a factual consistency of the insight data.

In some embodiments, the guardrail application includes NeMo guardrail.

In some embodiments, the two or more AI modules includes a text processing AI module which may be configured for generating a summary data corresponding to a performance of the activity associated with the integrated biomechanical data. Further, the insight data includes the summary data.

In some embodiments, the two or more activity phases include a first activity phase associated with a first time period and a second activity phase associated with a second time period. Further, the second time period occurs later than the first time period. Further, the method 900 further includes generating, using the processing device 1104, a transition data based on each of the first activity phase and the second activity phase. Further, the transition data represents a transition from the first activity phase to the second activity phase. Further, the insight data includes the transition data.

In some embodiments, the transition data includes an inefficiency data representing an inefficiency in the performance of the activity in each of the two or more activity phases.

In some embodiments, the insight data includes an improvement data representing an improvement in the performance of the activity based on the integrated biomechanical data.

In some embodiments, the method 900 may further include computing, using the processing device 1104, a transition metric based on the transition data. Further, the transition metric corresponds to the transition associated with the performance of the activity.

In some embodiments, the transition includes one or more of a rotational transition and positional transition of the entity.

In some embodiments, the user includes a coach.

In some embodiments, the coach includes a shooting coach.

In some embodiments, the user includes a rehabilitation therapist.

In some embodiments, the method 900 may further include storing, using a storage device 1106, the integrated biomechanical data associated with the integrated biomechanical dataset.

In some embodiments, the method 900 may further include storing, using a storage device 1106, the insight data.

In some embodiments, the insight data includes a feedback data representing a feedback based on the integrated biomechanical data. Further, the two or more AI modules include a conversational artificial intelligence (AI) module. Further, the generating of the insight data includes generating the feedback data using the conversational AI module.

In some embodiments, the skeletal model data includes a characteristic data representing a characteristic associated with the skeletal model of the user.

In some embodiments, the characteristic data includes a joint position data representing a position of a joint with respect to a user body.

In some embodiments, the joint position data further represents a position of the joint with respect to an environment where the activity may be performed.

In some embodiments, the characteristic data includes a joint angle data representing an angle subtended by a body part of the user in relation to a user body.

In some embodiments, the characteristic data includes a joint movement data corresponding to a movement of a joint associated with the user.

In some embodiments, the joint includes one or more of a wrist joint, a shoulder joint, an ankle joint, a hip joint, and a knee joint.

In some embodiments, the user includes a performer associated with the activity. Further, the integrated biomechanical data includes a body pose estimation data representing one or more of an alignment and an orientation of a body part of the performer.

In some embodiments, the integrated biomechanical data includes an additional contextual data corresponding to an additional information associated with the activity. Further, the generating of the insight data may be based on the additional contextual data.

In some embodiments, the processing of the raw integrated biomechanical dataset further includes determining a key feature associated with the integrated biomechanical data.

In some embodiments, the activity includes two or more activity phases. Further, the integrated biomechanical data includes two or more integrated biomechanical data representing each of the plurality of activity phases. Further, the video data includes one or more of a video frame data and a video metadata. Further, the video frame data corresponds to a visual information captured in each of the two or more activity phases. Further, the video metadata corresponds to an additional information about the visual content associated with the activity.

In some embodiments, the additional information includes one or more of a video resolution and a video duration. Further, the video resolution corresponds to a resolution of the visual content. Further, the video duration corresponds to a duration of the visual content associated with the activity.

In some embodiments, the processing of the raw integrated biomechanical dataset further comprising normalizing one or more of the video frame data and the video metadata associated with the visual content associated with the activity.

In some embodiments, the additional contextual data includes an activity statistic data representing an information quantifying the performance of the activity.

In some embodiments, the activity statistic data includes one or more of an activity score data and an activity time data.

In some embodiments, the integrated biomechanical data includes a user role data representing a role of the user associated with the activity.

In some embodiments, the user device 1110 includes at least one of a mobile device, a desktop computer, and a laptop.

In some embodiments, the representation data may be generated based on a data representation module.

In some embodiments, the data representation module includes one or more of a vector store and a graph engine.

In some embodiments, the integrated biomechanical data includes an orientation data representing an orientation of the user based on a performance of the activity.

In some embodiments, the orientation data includes a rotational data representing a rotation associated with the user.

In some embodiments, the orientation data includes a positional data representing a position of the user based on an environment associated with the activity.

In some embodiments, the positional data further corresponds to the position of a body part of the user in relation to a user body.

In some embodiments, the key feature may be associated with one or more of reasoning activity and an exploration activity associated with the integrated biomechanical data.

In some embodiments, the integrated biomechanical data associated with the integrated biomechanical dataset includes a perspective 3D model data based on a performance of the activity. Further, the perspective 3D model data represents a perspective view of a 3D model of the object. Further, the object includes one or more of the user associated with the activity and an equipment associated with the activity.

In some embodiments, the perspective view corresponds to one or more of a right side view, a left side view, a top view, a bottom view, and a back side view of the 3D model of the object.

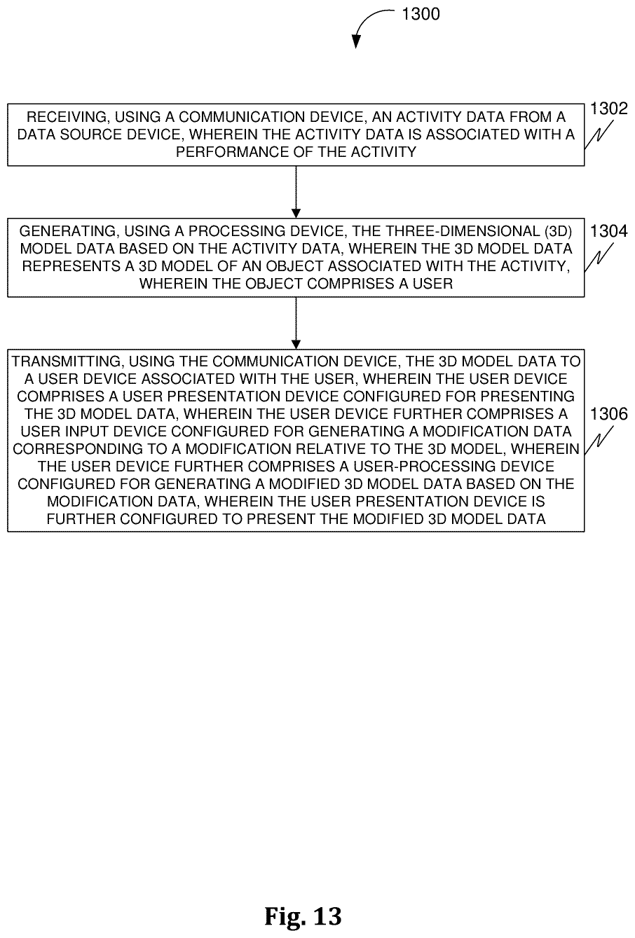

FIG. 13 illustrates a flowchart of a method 1300 for provisioning a three-dimensional model data based on an activity, in accordance with some embodiments. Accordingly, the method 1300 may include a step 1302 of receiving, using a communication device 1602, an activity data from a data source device 1606. Further, the activity data may be associated with a performance of the activity. Further, the method 1300 may include a step 1304 of generating, using a processing device 1604, the three-dimensional (3D) model data based on the activity data. Further, the 3D model data represents a 3D model of an object associated with the activity. Further, the object includes a user. Further, the method 1300 may include a step 1306 of transmitting, using the communication device 1602, the 3D model data to a user device 1608 associated with the user. Further, the user device 1608 includes a user presentation device 1700 which may be configured for presenting the 3D model data. Further, the user device 1608 further includes a user input device which may be configured for generating a modification data corresponding to a modification relative to the 3D model. Further, the user device 1608 further includes a user-processing device which may be configured for generating a modified 3D model data based on the modification data. Further, the user presentation device 1700 may be further configured to present the modified 3D model data.

In some embodiments, the object further includes an equipment associated with the activity. Further, the 3D model data includes one or more of a user model data representing the 3D model of the user associated with the activity and an equipment model data corresponding to the 3D model of the equipment.

In some embodiments, the user model data includes a skeletal model data representing a skeletal model of the user. Further, the skeletal model includes two or more line segments. Further, each of the two or more line segments includes two endpoints. Further, the skeletal model further includes two or more nodes. Further, each of the two or more nodes connects at least two of the two or more line segments. Further, each of the two or more line segments and the two or more nodes may be connected based on an anatomical structure of the user.

In some embodiments, the modification data includes a skeletal adjustment data corresponding to an adjustment performed on the skeletal model of the user. Further, the adjustment corresponds to a change in one or more of the characteristic associated with the user model.

In some embodiments, the user includes each of an athlete and a coach. Further, the user device 1608 includes an athlete device associated with the athlete and a coach device associated with the coach. Further, the activity may be performed by the athlete. Further, transmitting of the 3D model data to the user device 1608 includes transmitting the 3D model data to the coach device.

In some embodiments, the 3D model data includes a plurality of 3D model data. Further, each of the plurality of 3D model data may be associated with an identification data representing an identifier for each of the plurality of 3D model data.

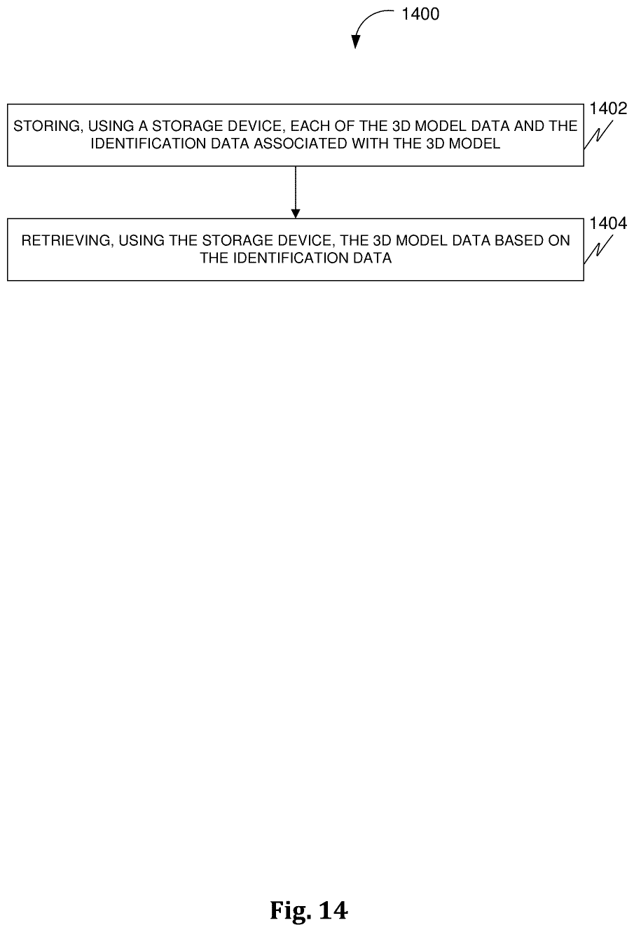

FIG. 14 illustrates a flowchart of a method 1400 for provisioning a three-dimensional model data based on an activity including retrieving, using the storage device, the 3D model data, in accordance with some embodiments. Further, in some embodiments, the method 1400 may include a step 1402 of storing, using a storage device, each of the 3D model data and the identification data associated with the 3D model. Further, in some embodiments, the method 1400 may include a step 1404 of retrieving, using the storage device, the 3D model data based on the identification data.

In some embodiments, the activity data includes a video data corresponding to a visual content associated with the activity. Further, the video data includes two or more video frames. Further, each of the two or more video frames may be associated with two or more time instances. Further, the feedback data includes two or more feedback data corresponding to the two or more time instances. Further, the video data includes two or more video frame data corresponding to the two or more video frames.

In some embodiments, the activity may be associated with two or more objects. Further, the two or more objects may be associated with two or more object data. Further, the two or more object data includes one or more of two or more user data and two or more equipment data. Further, the user data may be associated with the user and the equipment data corresponds to an equipment associated with the activity. Further, the two or more user data includes two or more user identifiers. Further, the two or more equipment data includes two or more equipment identifiers.

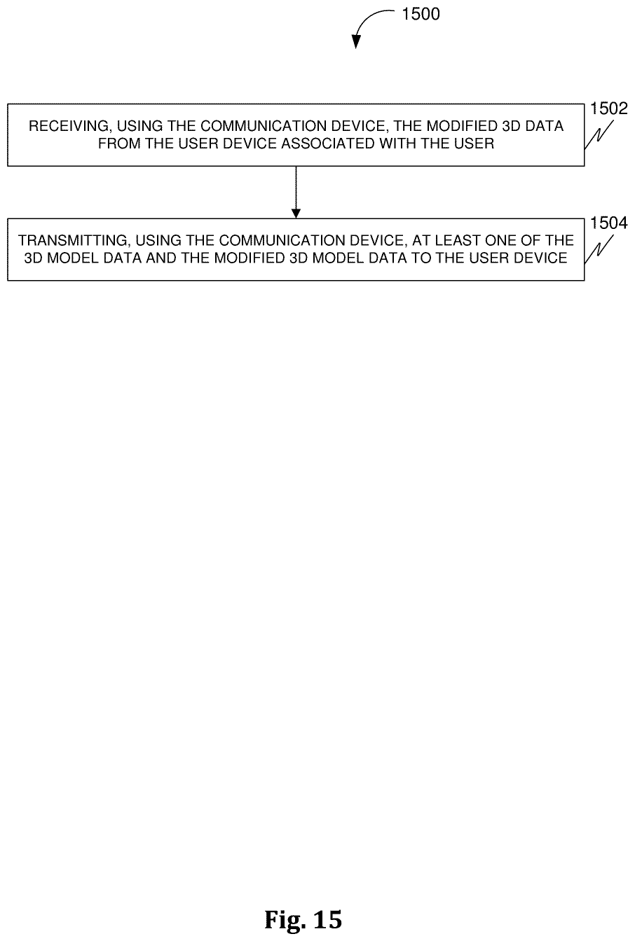

FIG. 15 illustrates a flowchart of a method 1500 for provisioning a three-dimensional model data based on an activity including receiving, using the communication device 1602, the modified 3D data from the user device 1608 associated with the user, in accordance with some embodiments. Further, in some embodiments, the method 1500 may include a step 1502 of receiving, using the communication device 1602, the modified 3D data from the user device 1608 associated with the user. Further, in some embodiments, the method 1500 may include a step 1504 of transmitting, using the communication device 1602, one or more of the 3D model data and the modified 3D model data to the user device 1608.

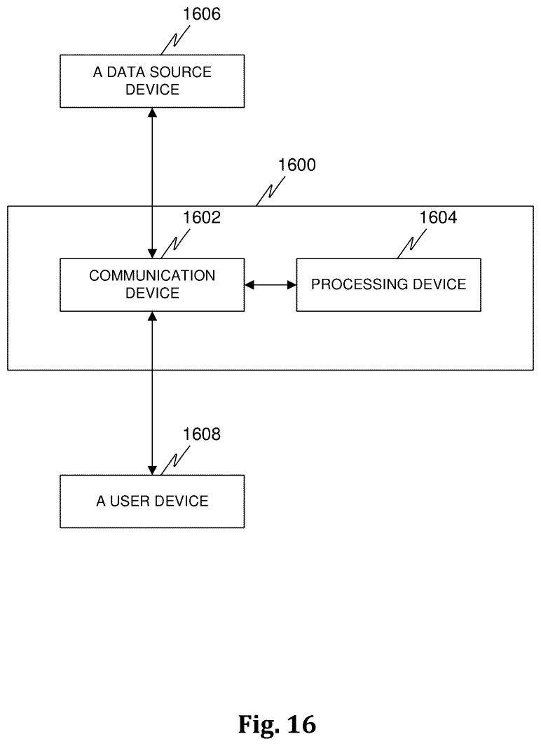

FIG. 16 illustrates a block diagram of a system 1600 for provisioning a three-dimensional model data based on an activity, in accordance with some embodiments. Accordingly, the system 1600 may include a communication device 1602. Further, the communication device 1602 may be configured for receiving an activity data from a data source device 1606. Further, the activity data may be associated with a performance of the activity. Further, the communication device 1602 may be configured for transmitting the 3D model data to a user device 1608 associated with the user. Further, the user device 1608 includes a user presentation device 1700 which may be configured for presenting the 3D model data. Further, the user device 1608 further includes a user input device which may be configured for generating a modification data corresponding to a modification relative to the 3D model. Further, the user device 1608 further includes a user-processing device which may be configured for generating a modified 3D model data based on the modification data. Further, the user presentation device 1700 may be further configured to present the modified 3D model data. Further, the system 1600 may include a processing device 1604 communicatively coupled with the communication device 1602. Further, the processing device 1604 may be configured for generating the three-dimensional (3D) model data based on the activity data. Further, the 3D model data represents a 3D model of an object associated with the activity. Further, the object includes a user.

In some embodiments, the object further includes an equipment associated with the activity. Further, the 3D model data includes one or more of a user model data representing the 3D model of the user associated with the activity and an equipment model data corresponding to the 3D model of the equipment.

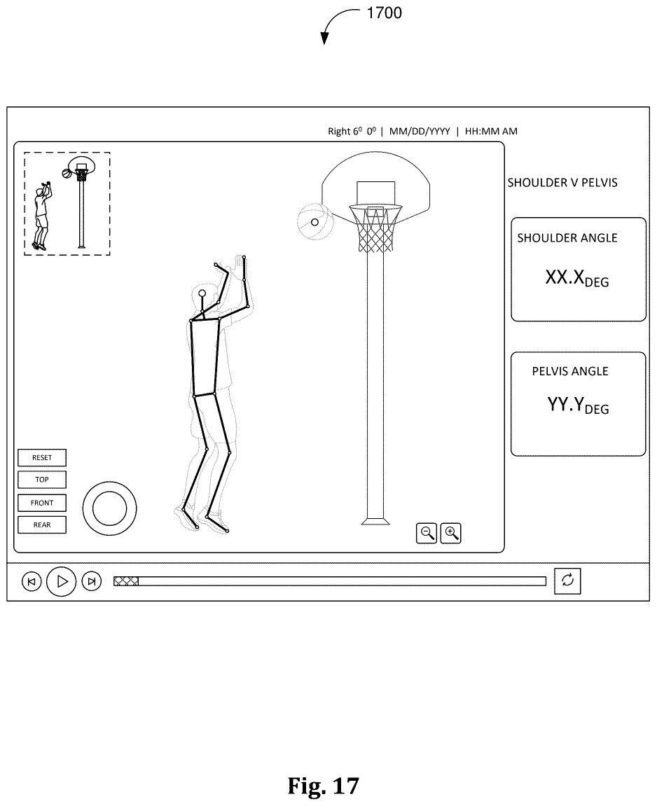

FIG. 17 illustrates a user presentation device 1700 presenting a real-time 3D skeleton rendering and adjustment tools during a collaborative coaching session, in accordance with some embodiments. Further, in some embodiments, the user model data includes a skeletal model data representing a skeletal model of the user. Further, the skeletal model includes two or more line segments. Further, each of the two or more line segments includes two endpoints. Further, the skeletal model further includes two or more nodes. Further, each of the two or more nodes connects at least two of the two or more line segments. Further, each of the two or more line segments and the two or more nodes may be connected based on an anatomical structure of the user.

In some embodiments, the modification data includes a skeletal adjustment data corresponding to an adjustment performed on the skeletal model of the user. Further, the adjustment corresponds to a change in one or more of the characteristic associated with the user model.

In some embodiments, the user includes each of an athlete and a coach. Further, the user device 1608 includes an athlete device associated with the athlete and a coach device associated with the coach. Further, the activity may be performed by the athlete. Further, the transmitting of the 3D model data to the user device 1608 includes transmitting the 3D model data to the coach device.

In some embodiments, the 3D model data includes a plurality of 3D model data. Further, each of the plurality of 3D model data may be associated with an identification data representing an identifier for each of the plurality of 3D model data.

Further, in some embodiments, the system 1600 may include a storage device communicatively coupled with the processing device 1604. Further, the storage device may be configured for storing each of the 3D model data and the identification data associated with the 3D model. Further, the storage device may be configured for retrieving the 3D model data based on the identification data.

In some embodiments, the activity data includes a video data corresponding to a visual content associated with the activity. Further, the video data includes two or more video frames. Further, each of the two or more video frames may be associated with two or more time instances. Further, the feedback data includes two or more feedback data corresponding to the two or more time instances. Further, the video data includes two or more video frame data corresponding to the two or more video frames.

In some embodiments, the activity may be associated with two or more objects. Further, the two or more objects may be associated with two or more object data. Further, the two or more object data includes one or more of two or more user data and two or more equipment data. Further, the user data may be associated with the user and the equipment data corresponds to an equipment associated with the activity. Further, the two or more user data includes two or more user identifiers. Further, the two or more equipment data includes two or more equipment identifiers.

Further, in some embodiments, the communication device 1602 may be further configured for receiving the modified 3D data from the user device 1608 associated with the user. Further, the communication device 1602 may be further configured for transmitting one or more of the 3D model data and the modified 3D model data to the user device 1608.

In some embodiments, the generating of the 3D model data includes generating the 3D model data based on a rendering framework.

In some embodiments, the rendering framework includes one or more of a Babylon.js framework, a Three.js framework, a PixiJS framework, an A-Frame framework, a Unity framework, and an Unreal Engine.

In some embodiments, the activity data includes two or more equipment data representing two or more equipment.

In some embodiments, the skeletal model data includes a characteristic data representing a characteristic associated with the skeletal model of the user.

In some embodiments, the characteristic data includes a joint position data representing a position of a joint relative to a user body.

In some embodiments, the joint position data further corresponds to a position of the joint relative to an environment where the activity may be performed.

In some embodiments, the characteristic data includes a joint angle data representing an angle subtended by a body part of the user with a user body.

In some embodiments, the characteristic data includes a joint movement data representing a movement of a joint associated with the user.

In some embodiments, the joint includes one or more of a wrist joint, a shoulder joint, an ankle joint, a hip joint, and a knee joint.

In some embodiments, the skeletal adjustment data includes an angle adjustment data representing the adjustment performed on an angular characteristic of the skeletal model.

In some embodiments, the skeletal adjustment data includes a flexion data representing one or more of an upward movement and a downward movement associated with the skeletal model of the user.

In some embodiments, the flexion data includes a knee flexion data representing a knee movement associated with the skeletal model of the user.

In some embodiments, the flexion data further includes a hip flexion data representing a hip movement associated with the skeletal model of the user.

In some embodiments, the flexion data further includes an ankle flexion data representing an ankle movement associated with the skeletal model of the user.

In some embodiments, the skeletal adjustment data includes a rotational adjustment data representing the adjustment performed on the rotational characteristic of the skeletal model of the user.

In some embodiments, the rotational adjustment data includes a shoulder rotational adjustment data representing the adjustment performed on a shoulder rotational characteristic of the skeletal model of the user.

In some embodiments, the rotational adjustment data includes an arm rotational adjustment data representing the adjustment performed on an arm rotational characteristic of the skeletal model of the user.

In some embodiments, the modification data includes a center of balance adjustment data representing the adjustment performed on a center of balance characteristic of the skeletal model of the user.

In some embodiments, the modification data further includes an equipment adjustment data representing an adjustment performed on the 3D model of the equipment based on the activity. Further, the adjustment corresponds to a change in one or more of the characteristic associated with the 3D model of the equipment.

In some embodiments, the modification data further includes a grip adjustment data representing the adjustment of a grip of the equipment in relation to the user based on the activity.

In some embodiments, each of the receiving and the transmitting may be performed based on a real-time communication protocol.

In some embodiments, the real-time communication protocol includes one or more of a WebRTC protocol, a WebSocket Protocol, a real-time transport protocol, a real-time messaging protocol, a VoI protocol, an XMP protocol, an MQTT protocol, and an HLS protocol.

In some embodiments, the method 1300 may further include receiving, using the communication device 1602, a coaching data corresponding to a coaching session from the coach device. Further, the receiving may be based on the real-time communication protocol. Further, the generating of the 3D model data may be further based on the coaching data. Further, the transmitting of the 3D model data to the user device 1608 includes transmitting the 3D model data to the athlete device.

In some embodiments, the athlete includes a first athlete and a second athlete. Further, the athlete device includes each of a first athlete device associated with the first athlete and a second athlete device associated with the second athlete. Further, the activity may be performed by the first athlete. Further, the transmitting of the 3D model data to the user device 1608 includes transmitting the 3D model data to the second athlete device.

In some embodiments, the modification data includes an athlete modification data representing the modification performed by the second athlete.

In some embodiments, the method 1300 may further include transmitting, using the communication device 1602, the modified 3D model data to the first athlete device associated with the first athlete.

In some embodiments, the coach includes a first coach and a second coach. Further, the coach device includes each of a first coach device associated with the first coach and a second coach device associated with the second coach. Further, the activity may be performed by the athlete. Further, the transmitting of the 3D model data to the user device 1608 includes transmitting the 3D model data to one or more of the first coach device and the second coach device.

In some embodiments, the modification data includes a coach modification data corresponding to the modification performed by one or more of the first coach and the second coach.

In some embodiments, the athlete device may be associated with an athlete location, and the coach device may be associated with a coach location. Further, each of the athlete location and the coach location may be geographically distinct.

### In Some Embodiments, the User Includes a Team.

In some embodiments, the 3D model data further includes a performance data representing a performance of the user.

In some embodiments, the 3D model data includes a feedback data representing a feedback on the performance of the activity. Further, the 3D model data further includes the video data embedded with the feedback data.

In some embodiments, the method 1300 may further include analyzing, using the processing device 1604, the activity data. Further, the analyzing of the activity data further includes identifying each of the two or more user data and the two or more equipment data in the two or more video frame data.

In some embodiments, the 3D model data includes the video data overlaid with the feedback data.

In some embodiments, the 3D model data includes an enhanced video data based on an integration of the feedback data into the video data.

In some embodiments, the feedback data further includes an instruction data representing an instruction for the performance of the activity.

### In Some Embodiments, the User Includes a Participant.

In some embodiments, the participant includes two or more participants. Further, each of the two or more participants corresponds to one or more of two or more locations.

In some embodiments, the two or more participants include one or more of a first participant associated with a first location and a second participant associated with a second location. Further, each of the first location and the second location may be geographically distinct.

In some embodiments, the transmitting of the 3D model data to the user device 1608 includes broadcasting one or more of the 3D model data and the modified 3D model data based on a real-time synchronization platform.

In some embodiments, the real-time synchronization platform includes a distributed messaging platform.

In some embodiments, the distributed messaging platform includes one or more of a Redis and a Kafka.

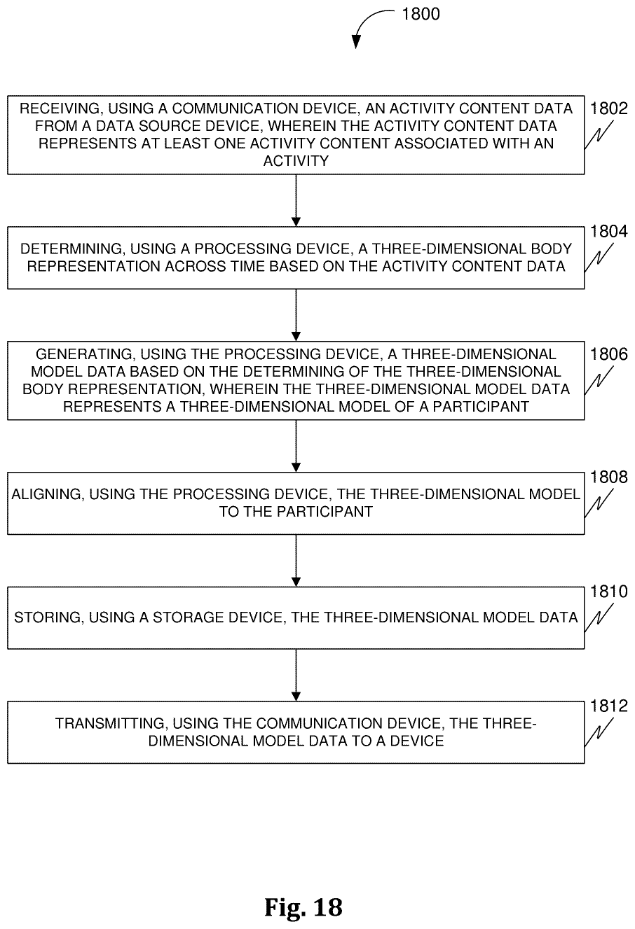

FIG. 18 illustrates a flowchart of a method 1800 for provisioning a three-dimensional model, in accordance with some embodiments. Accordingly, the method 1800 may include a step 1802 of receiving, using a communication device 1902, an activity content data from a data source device. Further, the activity content data represents one or more activity contents associated with an activity. Further, the method 1800 may include a step 1804 of determining, using a processing device 1904, a three-dimensional body representation across time based on the activity content data. Further, the method 1800 may include a step 1806 of generating, using the processing device 1904, a three-dimensional model data based on the determining of the three-dimensional body representation. Further, the three-dimensional model data represents a three-dimensional model of a participant. Further, the method 1800 may include a step 1808 of aligning, using the processing device 1904, the three-dimensional model to the participant. Further, the method 1800 may include a step 1810 of storing, using a storage device 1906, the three-dimensional model data. Further, the method 1800 may include a step 1812 of transmitting, using the communication device 1902, the three-dimensional model data to a device.

In some embodiments, the three-dimensional model includes one or more of a parametric body model, a mesh, a rigged skeleton, and a parametric avatar of the participant.

In some embodiments, the aligning of the three-dimensional model to the participant includes one or more of scaling the three-dimensional model relative to the participant and calibrating the three-dimensional model relative to the participant based on one or more of an anthropometric constraint and a skeletal constraint.

In some embodiments, the method 1800 may further include determining, using the processing device 1904, one or more model characteristics based on the three-dimensional body representation. Further, the generating of the three-dimensional model data may be further based on the one or more model characteristics.

In some embodiments, the one or more model characteristics include one or more of a bone-length constraint and a joint limit associated with the three-dimensional model.

In some embodiments, the three-dimensional model data may be associated with a file format. Further, the file format may be one or more of a GLB format and a FBX format.

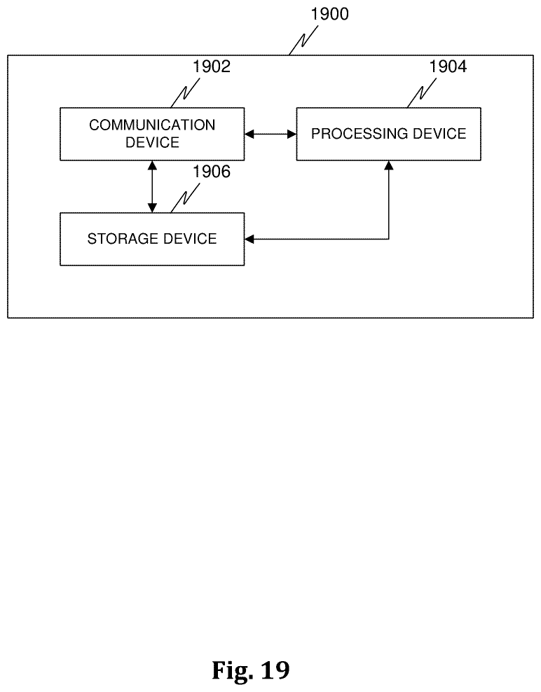

FIG. 19 illustrates a block diagram of a system 1900 for provisioning a three-dimensional model, in accordance with some embodiments. Accordingly, the system 1900 may include a communication device 1902. Further, the communication device 1902 may be configured for receiving an activity content data from a data source device. Further, the activity content data represents one or more activity contents associated with an activity. Further, the communication device 1902 may be configured for transmitting a three-dimensional model data to a device. Further, the system 1900 may include a processing device 1904 communicatively coupled with the communication device 1902. Further, the processing device 1904 may be configured for determining a three-dimensional body representation across time based on the activity content data. Further, the processing device 1904 may be configured for generating the three-dimensional model data based on the determining of the three-dimensional body representation. Further, the three-dimensional model data represents a three-dimensional model of a participant. Further, the processing device 1904 may be configured for aligning the three-dimensional model to the participant. Further, the system 1900 may include a storage device 1906 communicatively coupled with the processing device 1904. Further, the storage device 1906 may be configured for storing the three-dimensional model data.

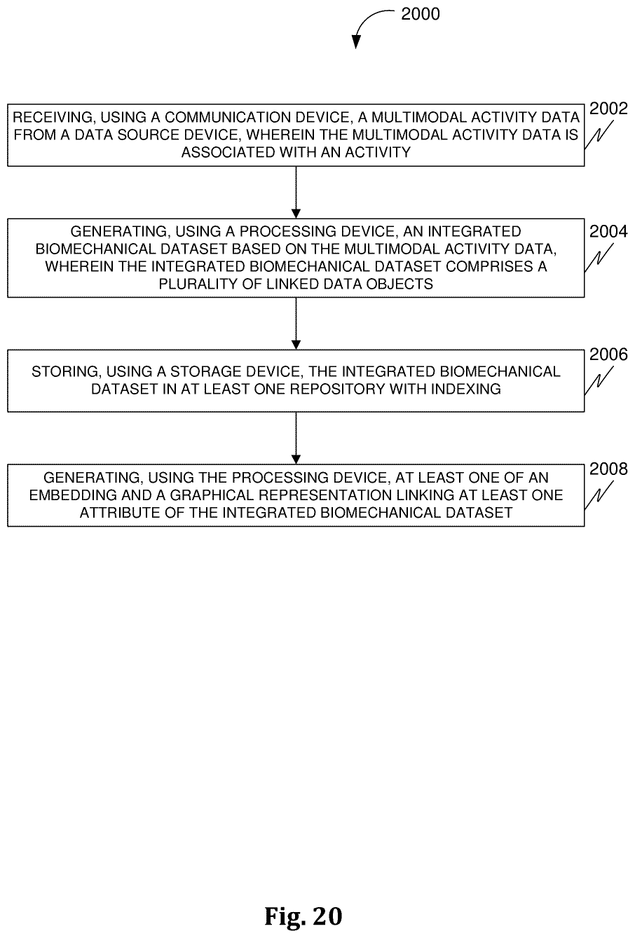

FIG. 20 illustrates a flowchart of a method 2000 for facilitating dataset creation and indexing, in accordance with some embodiments. Accordingly, the method 2000 may include a step 2002 of receiving, using a communication device 2302, a multimodal activity data from a data source device. Further, the multimodal activity data may be associated with an activity. Further, the method 2000 may include a step 2004 of generating, using a processing device 2304, an integrated biomechanical dataset based on the multimodal activity data. Further, the integrated biomechanical dataset includes two or more linked data objects. Further, the method 2000 may include a step 2006 of storing, using a storage device 2306, the integrated biomechanical dataset in one or more repositories with indexing. Further, the method 2000 may include a step 2008 of generating, using the processing device 2304, one or more of an embedding and a graphical representation linking one or more attributes of the integrated biomechanical dataset.

In some embodiments, each of the embedding and the graphical representation enables a retrieval of one or more time-aligned dataset portions associated with the integrated biomechanical dataset.

In some embodiments, the multimodal activity data includes one or more of a video data, a pose data, a metric/heuristic data, a sensor data, and a metadata associated with the activity.

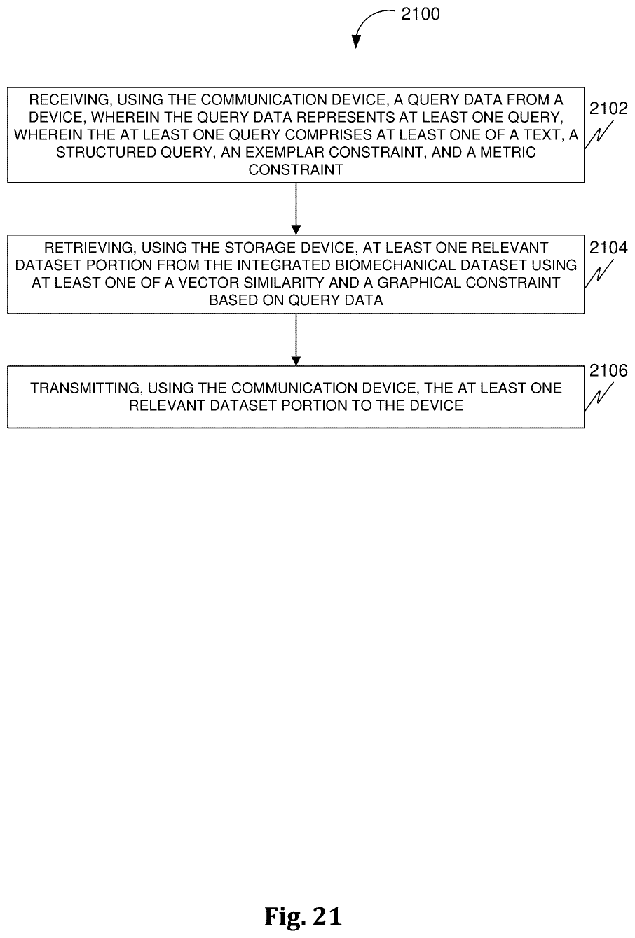

FIG. 21 illustrates a flowchart of a method 2100 for facilitating dataset creation and indexing including retrieving, using the storage device 2306, at least one relevant dataset portion from the integrated biomechanical dataset using at least one of a vector similarity and a graphical constraint, in accordance with some embodiments. Further, in some embodiments, the method 2100 may include a step 2102 of receiving, using the communication device 2302, a query data from a device. Further, the query data represents one or more queries. Further, the one or more queries include one or more of a text, a structured query, an exemplar constraint, and a metric constraint. Further, in some embodiments, the method 2100 may include a step 2104 of retrieving, using the storage device 2306, one or more relevant dataset portions from the integrated biomechanical dataset using one or more of a vector similarity and a graphical constraint based on query data. Further, in some embodiments, the method 2100 may include a step 2106 of transmitting, using the communication device 2302, the one or more relevant dataset portions to the device.

In some embodiments, the one or more relevant dataset portions includes one or more of a segment, a metric, and a three-dimensional asset relative to the activity.

In some embodiments, the one or more attributes may be one or more of an entity and a segment associated with the integrated biomechanical dataset.

In some embodiments, the method 2100 may further include generating, using the processing device 2304, a feedback data based on the one or more relevant dataset portions. Further, the feedback data represents one or more feedbacks. Further, the transmitting of the one or more relevant dataset portions includes transmitting of the feedback data to the device.

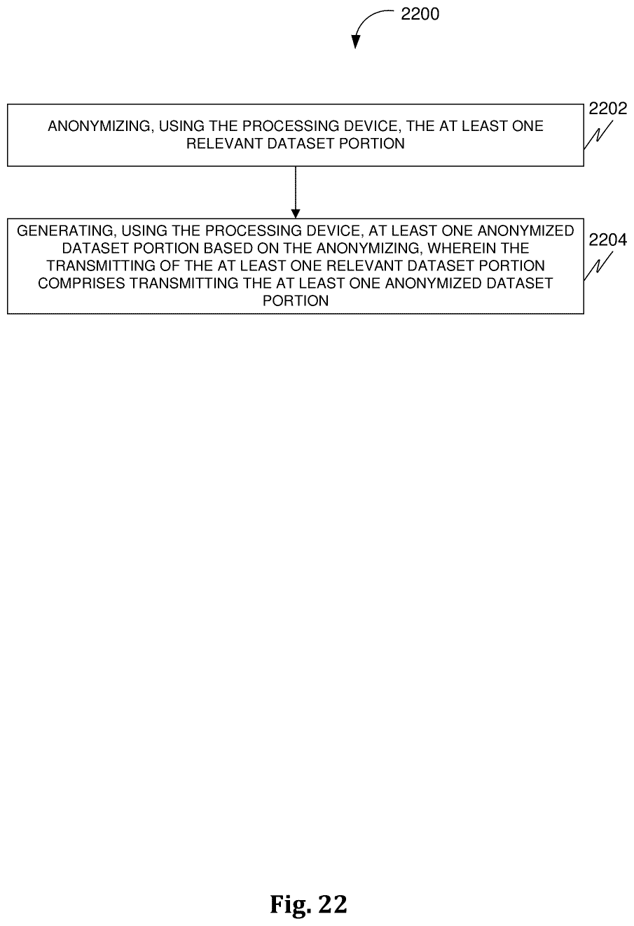

FIG. 22 illustrates a flowchart of a method 2200 for facilitating dataset creation and indexing including generating, using the processing device 2304, at least one anonymized dataset portion, in accordance with some embodiments. Further, in some embodiments, the method 2200 may include a step 2202 of anonymizing, using the processing device 2304, the one or more relevant dataset portions. Further, in some embodiments, the method 2200 may include a step 2204 of generating, using the processing device 2304, one or more anonymized dataset portions based on the anonymizing. Further, the transmitting of the one or more relevant dataset portions includes transmitting the one or more anonymized dataset portions.

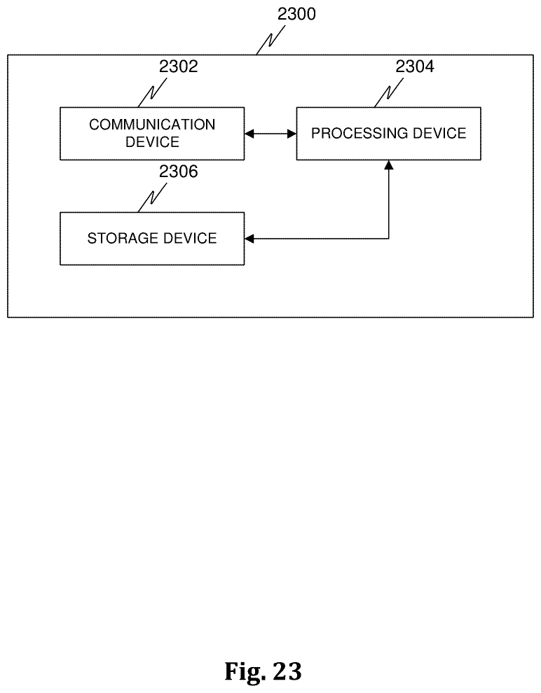

FIG. 23 illustrates a block diagram of a system 2300 for facilitating dataset creation and indexing, in accordance with some embodiments. Accordingly, the system 2300 may include a communication device 2302 which may be configured for receiving a multimodal activity data from a data source device. Further, the multimodal activity data may be associated with an activity. Further, the system 2300 may include a processing device 2304 communicatively coupled with the communication device 2302. Further, the processing device 2304 may be configured for generating an integrated biomechanical dataset based on the multimodal activity data. Further, the integrated biomechanical dataset includes two or more linked data objects. Further, the processing device 2304 may be configured for generating one or more of an embedding and a graphical representation linking one or more attributes of the integrated biomechanical dataset. Further, the system 2300 may include a storage device 2306 communicatively coupled with the processing device 2304. Further, the storage device 2306 may be configured for storing the integrated biomechanical dataset in one or more repositories with indexing.

Although the invention has been explained in relation to its preferred embodiment, it is to be understood that many other possible modifications and variations can be made without departing from the spirit and scope of the invention as hereinafter claimed.

What is claimed is:

1. A method of provisioning a feedback based on an activity, the method comprising:
receiving, using a communication device, an activity data associated with a performance of the activity from a data source device, wherein the receiving is performed based on a real-time communication protocol;
generating, using a processing device, a feedback data using an artificial intelligence (AI) module based on the activity data;
transmitting, using the communication device, the feedback data to a user device associated with a user, wherein the transmitting is performed based on the real-time communication protocol.

2. The method of claim 1, wherein the activity data comprises a video data representing a visual content associated with the activity.

3. The method of claim 1, wherein the activity is associated with a plurality of objects, wherein the feedback data comprises an object data representing an object associated with the activity, wherein the object is a sport equipment.

4. The method of claim 1, wherein the user comprises at least one of an athlete and a coach, wherein the user device comprises an athlete device associated with the athlete and a coach device associated with the coach, wherein the athlete comprises at least one of a first athlete and a second athlete, wherein the athlete device comprises at least one of a first athlete device associated with the first athlete and a second athlete device associated with the second athlete, wherein the performance of the activity comprises performance of the activity by the first athlete, wherein the transmitting of the feedback data to the user device comprises transmitting the feedback data to the coach device in a first time period, wherein the method further comprises receiving, using the communication device, at least one of a coaching data from the coach device and an athlete data from the second athlete device, wherein the coaching data represents a coaching session, wherein the athlete data represents a peer feedback from the second athlete, wherein the receiving is based on the real-time communication protocol, wherein the generating of the feedback data is further based on at least one of the coaching data and the athlete data, wherein the transmitting of the feedback data to the user device further comprises transmitting the feedback data to the athlete device in a second time period, wherein the second time period occurs later than the first time period.

5. The method of claim 1, wherein the activity data comprises a sensor data associated with a sensor, wherein the sensor data represents a variable attribute associated with the activity, wherein the sensor comprises at least one of a wearable sensor configured to be worn by the user and an an external sensor placed in an environment associated with the user, wherein the variable attribute comprises a user-based attribute associated with the user relative to the activity.

6. The method of claim 2, wherein the feedback data comprises an insight data corresponding to an insight on the performance of the activity, wherein the feedback data further comprises the video data embedded with the insight data, wherein the video data comprises a plurality of video frames, wherein each of the plurality of video frames is associated with a plurality of time instances associated with the activity, wherein the insight data comprises a plurality of insight data corresponding to the plurality of time instances.

7. The method of claim 6, wherein the feedback data comprises at least one of a scorecard data, an overlaid video data, and an enhanced video data, wherein the scorecard data represents a score associated with the activity, wherein the overlaid video data represents the video data overlaid with the insight data, wherein the enhanced video data represents an integration of the insight data into the video data.

8. The method of claim 1, wherein the activity data comprises a plurality of activity data corresponding to a plurality of activity phases associated with the activity, wherein the plurality of activity data comprises a first activity data representing a first activity phase associated with a first time period and a second activity data representing a second activity phase associated with a second time period, wherein the second time period occurs later than the first time period, wherein the method further comprises determining, using the processing device, a transition data representing at least one transition between the first activity phase and the second activity phase, wherein the generating of feedback data is further based on the transition data, wherein the plurality of activity phases comprises a shot phase associated with the activity, wherein the shot phase comprises at least one of a transfer phase, a pocket phase and a release phase.

9. The method of claim 8, wherein the activity data further comprises a plurality of phase metrics data corresponding to each of the plurality of activity phases, wherein the plurality of phase metrics data comprises at least one of a reference phase metric data and a current phase metric data, wherein the reference phase metric data represents a standard metric value for the performance of the activity, wherein the current phase metric data represents a metric value generated based on the performance of the activity, wherein the method further comprises analyzing, using the processing device, each of the reference phase metric data and the current phase metric data, wherein the generating of the feedback data is further based on analyzing of the reference phase metric data and the current phase metric data.

10. The method of claim 1, wherein the activity data comprises an activity factor data representing a factor affecting the performance by the user, wherein the activity factor data comprises a first activity factor data associated with a first time period and a second activity factor data associated with a second time period, wherein the second time period occurs later than the first time period, wherein the method further comprises generating, using the processing device, an adaptive score data based on each of the first activity factor data and the second activity factor data, wherein the adaptive score data represents a score associated with the performance of the activity, wherein the feedback data comprises the adaptive score data.

11. A system of provisioning a feedback based on an activity, the system comprising:
a communication device configured for:
receiving an activity data associated with a performance of the activity from a data source device, wherein the receiving is performed based on a real-time communication protocol; and
transmitting a feedback data to a user device associated with a user, wherein the transmitting is performed based on the real-time communication protocol; and
a processing device communicatively coupled with the communication device, wherein the processing device is configured for generating the feedback data using an artificial intelligence (AI) module based on the activity data.

12. The system of claim 11, wherein the activity data comprises a video data representing a visual content associated with the activity.

13. The system of claim 11, wherein the activity is associated with a plurality of objects, wherein the feedback data comprises an object data representing an object associated with the activity, wherein the object is a sport equipment.

14. The system of claim 11, wherein the user comprises at least one of an athlete and a coach, wherein the user device comprises an athlete device associated with the athlete and a coach device associated with the coach, wherein the athlete comprises at least one of a first athlete and a second athlete, wherein the athlete device comprises at least one of a first athlete device associated with the first athlete and a second athlete device associated with the second athlete, wherein the performance of the activity comprises performance of the activity by the first athlete, wherein the transmitting of the feedback data to the user device comprises transmitting the feedback data to the coach device in a first time period, wherein the communication device is further configured for receiving at least one of a coaching data from the coach device and an athlete data from the second athlete device, wherein the coaching data represents a coaching session, wherein the athlete data represents a peer feedback from the second athlete, wherein the receiving is based on the real-time communication protocol, wherein the generating of the feedback data is further based on at least one of the coaching data and the athlete data, wherein the transmitting of the feedback data to the user device further comprises transmitting the feedback data to the athlete device in a second time period, wherein the second time period occurs later than the first time period.

15. The system of claim 11, wherein the activity data comprises a sensor data associated with a sensor, wherein the sensor data represents a variable attribute associated with the activity, wherein the sensor comprises at least one of a wearable sensor configured to be worn by the user and an an external sensor placed in an environment associated with the user, wherein the variable attribute comprises a user-based attribute associated with the user relative to the activity.

16. The system of claim 12, wherein the feedback data comprises an insight data corresponding to an insight on the performance of the activity, wherein the feedback data further comprises the video data embedded with the insight data, wherein the video data comprises a plurality of video frames, wherein each of the plurality of video frames is associated with a plurality of time instances associated with the activity, wherein the insight data comprises a plurality of insight data corresponding to the plurality of time instances.

17. The system of claim 16, wherein the feedback data comprises at least one of a scorecard data, an overlaid video data, and an enhanced video data, wherein the scorecard data represents a score associated with the activity, wherein the overlaid video data represents the video data overlaid with the insight data, wherein the enhanced video data represents an integration of the insight data into the video data.

18. The system of claim 11, wherein the activity data comprises a plurality of activity data corresponding to a plurality of activity phases associated with the activity, wherein the plurality of activity data comprises a first activity data representing a first activity phase associated with a first time period and a second activity data representing a second activity phase associated with a second time period, wherein the second time period occurs later than the first time period, wherein the processing device is further configured for determining a transition data representing at least one transition between the first activity phase and the second activity phase, wherein the generating of feedback data is further based on the transition data, wherein the plurality of activity phases comprises a shot phase associated with the activity, wherein the shot phase comprises at least one of a transfer phase, a pocket phase and a release phase.

19. The system of claim 18, wherein the activity data further comprises a plurality of phase metrics data corresponding to each of the plurality of activity phases, wherein the plurality of phase metrics data comprises at least one of a reference phase metric data and a current phase metric data, wherein the reference phase metric data represents a standard metric value for the performance of the activity, wherein the current phase metric data represents a metric value generated based on the performance of the activity, wherein the processing device is further configured for analyzing each of the reference phase metric data and the current phase metric data, wherein the generating of the feedback data is further based on analyzing of the reference phase metric data and the current phase metric data.

20. The system of claim 11, wherein the activity data comprises an activity factor data representing a factor affecting the performance by the user, wherein the activity factor data comprises a first activity factor data associated with a first time period and a second activity factor data associated with a second time period, wherein the second time period occurs later than the first time period, wherein the processing device is further configured for generating an adaptive score data based on each of the first activity factor data and the second activity factor data, wherein the adaptive score data represents a score associated with the performance of the activity, wherein the feedback data comprises the adaptive score data.
